(19) United States
(12) Patent Application Publication
(10) Pub. No.: US 2006/0003976 A1
(43) Pub. Date: Jan. 5, 2006

(54) Cholesterol/bile acid/bile acid derivative-modified therapeutic drug compounds

(76) Inventors: Yuehua Zhang, Bothell, WA (US); Lynn C. Gold, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/145,537

(22) Filed: Jun. 3, 2005

Related U.S. Application Data
(60) Provisional application No. 60/577,257, filed on Jun. 4, 2004.
(60) Provisional application No. 60/685,373, filed on May 31, 2005.

(51) Int. Cl. A61K 31/58; C07J 43/00

(52) U.S. Cl. 514/176; 540/107

(57) ABSTRACT

Cholesterol-modified anti-cancer therapeutic drug compounds, bile-acid-modified anti-cancer therapeutic drug compounds, and bile-acid-derivative-modified anti-cancer therapeutic drug compounds; emulsion, microemulsion, and micelle formulations that include the compounds, methods for administering the compounds and formulations; and methods for treating cancer using the compounds and formulations.

### CROSS-REFERENCES TO RELATED APPLICATIONS

This application claims the benefit of U.S. Provisional Application No. 60/577,257, filed Jun. 4, 2004, and U.S. Provisional Application No. 60/______, filed May 31, 2005, Attorney Docket No. SNUS125340, entitled CHOLESTEROL/BILE ACID/BILE ACID DERIVATIVE-MODIFIED THERAPEUTIC DRUG COMPOUNDS.

### FIELD OF THE INVENTION

The present invention relates to new therapeutic drugs; compositions of the new therapeutic drugs; and uses of the new therapeutic drugs and compositions.

### BACKGROUND OF THE INVENTION

The ability to administer biologically effective drugs that are poorly soluble in biocompatible solvents to mammals has been a major hurdle in the realm of pharmaceutical and medicinal chemistry. In particular, difficulties arise when an active drug is either insoluble in water or unstable in other biocompatible solvents. Solubility problems are common and often cause delays in drug development. It is estimated that thirty percent of existing drugs are poorly soluble. Several technologies have been developed to facilitate the delivery of poorly soluble and insoluble compounds. Examples of technologies specifically designed to solve solubility problems include complexing agents, nanoparticles, microemulsions, solubility enhancing formulations, prodrugs and water soluble prodrugs, and novel polymer systems.

One way to improve the solubility of medicinal agents is to chemically modify them or conjugate them to another molecule to alter the solubility profile in a particular solvent. Conjugates of active drugs, often referred to as prodrugs, include a chemical derivative of a biologically-active parent compound. Prodrugs may be biologically inert or maintain activity that is substantially less than the parent or active compound. The parent compound is released from the prodrug in vivo by a variety of mechanisms, including, for example, hydrolysis or enzymatic cleavage. The rate of release is influenced by several factors, including the type of chemical bond joining the active parent drug to the conjugate moiety.

Potent drugs that are poorly soluble in water include camptothecin and its analogs, taxanes (e.g., paclitaxel, docetaxel), candesartan, amphotericin B, azathioprine, cyclosporine, entacapone, danazol, eletriptan, and bosentan, to name a few. There continues to be a need for new methods, which are both safe and effective, of solubilizing and delivering poorly soluble active drug molecules.

### SUMMARY OF THE INVENTION

In one aspect, the present invention provides therapeutic drug compounds that have been modified to increase their lipophilicity. The compounds of the invention include a therapeutic drug moiety and a lipophilic moiety. The therapeutic drug moiety is covalently coupled to the lipophilic moiety either directly or by a linker moiety. In one embodiment, the lipophilic moiety is derived from cholesterol or a cholesterol derivative. In one embodiment, the lipophilic moiety is derived from a bile acid or a bile acid derivative. In one embodiment, the therapeutic drug moiety is derived from an anti-cancer therapeutic drug, such as paclitaxel, docetaxel, and camptothecin. Methods for making the modified therapeutic drugs are also provided.

In one embodiment, the invention provides cholesterol-modified anti-cancer therapeutic drug compounds in which the cholesterol moiety is covalently coupled to the anti-cancer therapeutic drug moiety through a linker moiety. These cholesterol-modified anti-cancer therapeutic drug compounds have the following formula:

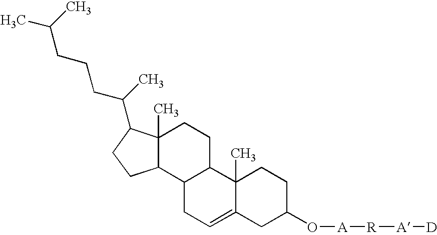

or a stereoisomer, tautomer, or pharmaceutically acceptable salt thereof, wherein

A and A′ are independently selected from the group consisting of

(a) —S(═O)—,

(b) —SO₂—,

(c) —C(═O)—

(d) —C(═O)O—,

(e) —C(═O)NR₁—,

(f) —C(═O)OC(═O)—,

(g) —P(═O)(OR₁)O—,

(h) —P(═O)(NR₁)O—,

(i) —SO₂O—,

(j) —S(═O)NR₁—, and

(k) —SO₂NR₁—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl;

R is a divalent radical selected from the group consisting of

(a) substituted or unsubstituted alkylene,

(b) substituted or unsubstituted heteroalkylene,

(c) substituted or unsubstituted cycloalkylene,

(d) substituted or unsubstituted arylene,

(e) amino acid,

(f) peptide,

(g) saccharide, and

(h) alkylene oxide oligomer; and

D is an anti-cancer therapeutic agent moiety.

In another embodiment, the invention provides cholesterol-modified anti-cancer therapeutic drug compounds having the formula:

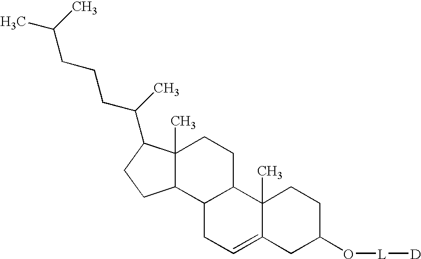

or a stereoisomer, tautomer, or pharmaceutically acceptable salt thereof, wherein L is selected from the group consisting of

(a) —S(═O)—,

(b) —SO₂—,

(c) —C(═O)—

(d) —C(═O)OC(═O)—,

(e) —P(═O)(OR₁)—, and

(f) —P(═O)(NR₁)—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl; and

D is an anti-cancer therapeutic agent moiety.

In another embodiment, the invention provides bile acid- and bile-acid-derivative-modified anti-cancer therapeutic drug compounds having the formula:

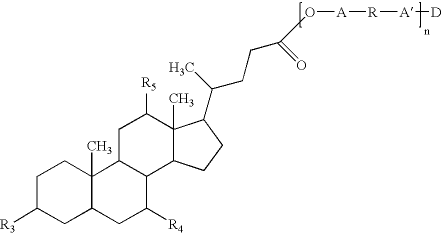

or a stereoisomer, tautomer, or pharmaceutically acceptable salt thereof, wherein

R₃ is OR₆ₐ, and R₄ and R₅ are H; or

R₃ is OR₆ₐ, R₄ is OR_{6b}, and R₅ is H; or

R₃ is OR₆ₐ, R₄ is OR_{6b}, and R₅ is OR_{6c},

wherein R₆ₐ, R_{6b}, and R_{6c} are independently selected from the group consisting of H, substituted or unsubstituted alkyl, substituted or unsubstituted aralkyl, and substituted and unsubstituted acyl;

n is 0 or 1;

A and A′ are independently selected from the group consisting of

(a) —S(═O)—,

(b) —SO₂—,

(c) —C(═O)—

(d) —C(═O)O—,

(e) —C(═O)NR₁—,

(f) —C(═O)OC(═O)—,

(g) —P(═O)(OR₁)O—,

(h) —P(═O)(NR₁)O—,

(i) —SO₂O—,

(j) —S(═O)NR₁—, and

(k) —SO₂NR₁—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl;

R is a divalent radical selected from the group consisting of

(a) substituted or unsubstituted alkylene,

(b) substituted or unsubstituted heteroalkylene,

(c) substituted or unsubstituted cycloalkylene,

(d) substituted or unsubstituted arylene,

(e) amino acid,

(f) peptide,

(g) saccharide, and

(h) alkylene oxide oligomer; and

D is an anti-cancer therapeutic agent moiety.

In certain of the embodiments above, the anti-cancer therapeutic agent moiety is selected from a paclitaxel moiety, docetaxel moiety, a camptothecin moiety, and derivatives thereof. Suitable anti-cancer therapeutic agent moieties include a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

In another aspect of the invention, compositions that include the compounds of the invention are provided. In one embodiment, the composition includes a compound of the invention, optionally one or more other therapeutic agents, and a lipophilic medium. Methods for making the compositions are also provided.

In a further aspect, the invention provides emulsion and micelle formulations that include a compound of the invention. The emulsion formulations include an oil phase and an aqueous phase. The emulsion may be an oil-in-water emulsion or a water-in-oil emulsion. The micelle formulation includes a compound of the invention and an aqueous phase. Methods for making the emulsion and micelle formulations are also provided.

In other aspects, methods for administering the compounds of the invention to a subject in need thereof, and methods for treating a condition treatable by administration of a compound of the invention are also provided.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates the chemical structures of cholesterol, cholic acid, chenodeoxycholic acid, and lithocholic acid;

FIG. 2 is a schematically illustrations reaction of cholesterol and a therapeutic drug containing a carboxyl group to provide a cholesterol-modified therapeutic drug compound;

FIG. 3 schematically illustrates cholesterol functionalization with a carboxyl group (—COOH), and reaction of the resulting acid with an appropriately functionalized therapeutic drug compound to provide a cholesterol-modified therapeutic drug compound; X may be a hydroxyl group, carboxyl group, mercapto or thiol group, or an amino group; Y may be an oxygen atom, a sulfur atom, an amino group, a substituted or unsubstituted alkylamino or cycloalkylamino group, a substituted or unsubstituted arylamino, or an aralkylamino group;

FIG. 4 schematically illustrates preparation of cholesterol succinate-10-(10-hydroxycamptothecin) and cholesterol succinate-10-(7-ethyl-10-hydroxycamptothecin);

FIG. 5 schematically illustrates preparation of cholesterol succinate-20-camptothecin;

FIG. 6 schematically illustrates preparations of lithocholic acid-modified 10-hydroxycamptothecin and lithocholic acid-modified 7-ethyl-10-hydroxycamptothecin;

FIG. 7 schematically illustrates preparation of lithocholic acid-modified camptothecin;

FIG. 8 schematically illustrates preparation of a lithocholic-acid-derivative-modified 10-hydroxycamptothecin and a lithocholic-acid-derivative-modified 7-ethyl-10-hydroxycamptothecin; R may be independently selected from n-alkyl, branched alkyl, substituted alkyl, cycloalkyl or substituted cycloalkyl, aryl or substituted aryl, aralkyl or substituted aralkyl, allyl or substituted allyl, benzyl or substituted benzyl, acyl, alkyl phosphate, alkyl phosphonate, aryl phosphate, aryl phosphonate, alkyl sulfate, alkyl sulfonate, aryl sulfate, and aryl sulfonate; and

FIG. 9 schematically illustrates preparation of a lithocholic-acid-derivative-modified camptothecin; R may be independently selected from n-alkyl, branched alkyl, substituted alkyl, cycloalkyl or substituted cycloalkyl, aryl or substituted aryl, aralkyl or substituted aralkyl, allyl or substituted allyl, benzyl or substituted benzyl, acyl, alkyl phosphate, alkyl phosphonate, aryl phosphate, aryl phosphonate, alkyl sulfate, alkyl sulfonate, aryl sulfate, and aryl sulfonate.

### DETAILED DESCRIPTION OF THE INVENTION

In one aspect, the present invention provides therapeutic drug compounds that have been modified to increase their lipophilicity. The compounds of the invention are modified therapeutic drugs. The compounds of the invention include a therapeutic drug moiety and a lipophilic moiety.

In some embodiments, the therapeutic drug moiety is covalently coupled to the lipophilic moiety through a linker moiety. In other embodiments, the therapeutic drug moiety is directly covalently coupled to the lipophilic moiety without a linker moiety.

In one aspect, the present invention provides modified therapeutic drug compounds that include a therapeutic drug moiety and a lipophilic moiety. In one embodiment, the lipophilic moiety is cholesterol. In one embodiment, the lipophilic moiety is a bile acid. In one embodiment, the lipophilic moiety is a bile-acid derivative.

In one embodiment, the modified therapeutic drug compound is a cholesterol-modified therapeutic drug compound, wherein a cholesterol moiety is covalently coupled to a therapeutic drug moiety.

In another embodiment, the modified therapeutic drug compound is a bile-acid-modified therapeutic drug compound, wherein a bile-acid moiety is covalently coupled to a therapeutic drug moiety.

In another embodiment, the modified therapeutic drug compound is a bile-acid-derivative-modified therapeutic drug compound, wherein a bile-acid-derivative moiety is covalently coupled to a therapeutic drug moiety.

As used herein, the term “modified therapeutic drug compound” refers to a therapeutic drug compound that has been modified to include a cholesterol moiety (i.e., to provide a cholesterol-modified therapeutic drug compound), a bile-acid moiety (i.e., to provide a bile-acid-modified therapeutic drug compound), or a bile-acid-derivative moiety (i.e., to provide a bile-acid-derivative-modified therapeutic drug compound). The covalent coupling of a cholesterol moiety, a bile acid moiety, or a bile-acid-derivative moiety to a therapeutic drug moiety can be direct or through a linker moiety. Methods for making the modified therapeutic drug compounds are also provided.

In another aspect of the invention, compositions that include one or more of the modified therapeutic drug compounds of the invention are provided. In one embodiment, the composition includes a lipophilic medium. In one embodiment, the lipophilic medium is a tocopherol. Methods for making the compositions are also provided.

In a further aspect, the invention provides emulsions that include one or more of the modified therapeutic drug compounds. In one embodiment, the emulsion includes a modified therapeutic drug compound, a lipophilic medium in which the modified therapeutic drug compound is soluble, and an aqueous medium. The emulsion may be an oil-in-water emulsion or a water-in-oil emulsion. In one embodiment, the lipophilic medium is a tocopherol. Methods for making the modified therapeutic drug compound-containing emulsions are also provided.

In another aspect, the invention provides micelle formulations that include one or more of the modified therapeutic drug compounds. In one embodiment, the micelle formulation includes a modified therapeutic drug compound, one or more solvents in which the modified therapeutic drug compound is soluble, one or more surfactants, and an aqueous medium.

In one embodiment, a modified therapeutic drug of the invention may be represented by formula (1):
B-A-R-A′-D 1
in which B is a cholesterol moiety, a bile-acid moiety, or a bile-acid-derivative moiety, A-R-A′ is a linker moiety, and D is a therapeutic drug moiety.

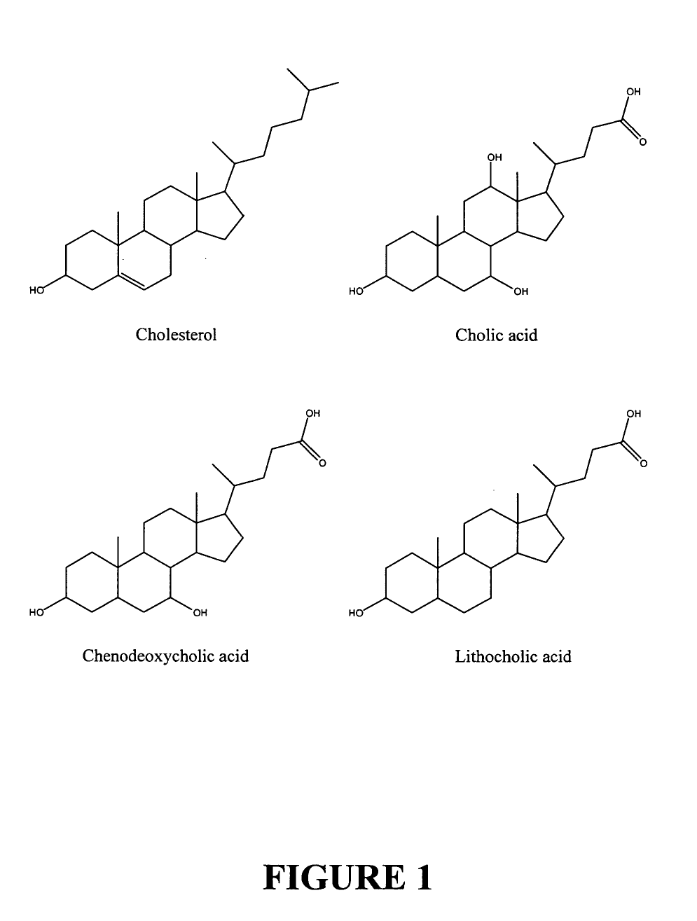

As used herein, the terms “cholesterol moiety,” “bile-acid moiety,” and “bile-acid derivative moiety” refer to a moieties derived from cholesterol, a bile acid, and a bile-acid-derivative, respectively, that can be covalently coupled to a therapeutic drug compound to provide a cholesterol-modified therapeutic drug compound, a bile-acid-modified therapeutic drug compound, and a bile-acid-derivative-modified therapeutic drug compound. The chemical structure of cholesterol is illustrated in FIG. 1. The chemical structures of three representative bile acids (i.e., cholic acid, chenodeoxycholic acid, and lithocholic acid) useful in making the modified therapeutic drug compounds of the invention are illustrated in FIG. 1. Suitable cholesterol, bile-acid, and bile-acid derivative moieties can be prepared from compounds illustrated in FIG. 1.

As used herein, “linker moiety” refers to an atom or a group of atoms that covalently link the cholesterol moiety, bile-acid moiety, or bile-acid-derivative moiety to a therapeutic drug moiety.

As used herein, the term “therapeutic drug moiety” refers to a therapeutic drug compound that can be covalently coupled to a cholesterol moiety, a bile-acid moiety, or a bile-acid-derivative moiety to provide a cholesterol-modified therapeutic drug compound, a bile-acid-modified therapeutic drug compound, or a bile-acid-derivative-modified therapeutic drug compound of the invention.

A cholesterol moiety, a bile-acid moiety, or a bile-acid-derivative moiety can be covalently coupled to a therapeutic drug moiety that has a reactive functional group, including, for example, a hydroxyl group (OH), an amino group (a primary amino group, NH₂, or secondary amino group, NHR), a mercapto or thiol group (SH), or carboxyl group (COOH).

In another embodiment, a modified therapeutic drug of the invention may be represented by formula (2):
B-D 2

In this embodiment, a cholesterol moiety, or a bile-acid moiety, or a bile-acid-derivative moiety (B) is directly covalently coupled to the therapeutic drug moiety (D) through a suitable bond, for example, an ester bond, ether bond, amide bond, anhydride bond, carbamate bond, carbonate bond, phosphate bond, phosphonate bond, or sulfate bond.

As noted above, a cholesterol moiety, a bile-acid moiety, or a bile-acid-derivative moiety can be covalently coupled to a therapeutic drug moiety that has a reactive functional group. Virtually any therapeutic drug compound having a suitable functional group, or that can be modified to include a suitable functional group, can be covalently coupled to a cholesterol moiety, a bile-acid moiety, or a bile-acid-derivative moiety to provide a cholesterol-modified therapeutic drug compound, a bile-acid-modified therapeutic drug compound, or a bile-acid-derivative-modified therapeutic drug compound of the invention, respectively. Representative functional groups include, for example, hydroxyl group (OH), amino group (primary amino group, NH₂, and secondary amino group, NHR), mercapto or thiol group (SH), carboxyl group (COOH), aldehyde group (—CH═O), oxiranyl group (—CH(O)CH₂), isocynato group (—N═C═O), sulfonyl chloride group (—SO₂Cl), sulfuric chloride group (—OSO₂Cl), phosphoryl chloride (—OPO₂Cl), allylic halide group, benzylic halide group, and substituted benzylic halide group. Therapeutic drug compounds that include the aforementioned functional groups are suitable for use in making the cholesterol-modified therapeutic drug compound, bile-acid-modified therapeutic drug compound, and bile-acid derivative-modified therapeutic drug compounds of the invention.

Therapeutic drug compounds selected for conjugation need not be substantially water-insoluble, although the cholesterol-modified therapeutic drug compounds, bile-acid-modified therapeutic drug compounds, and bile-acid-derivative-modified therapeutic drug compounds of the present invention are especially well suited for formulating and delivering such water-insoluble compounds. The modified therapeutic drug compounds of the invention provide for the solubilization of therapeutic drug compounds in pharmaceutical formulations that would be otherwise difficult to formulate for administration. The modified therapeutic drug compounds of the invention also provide for enhanced pharmacokinetic properties compared to the unmodified therapeutic drug compounds (i.e., parent compounds). For example, while some therapeutic drug compounds are rapidly cleared from a subject shortly after administration (e.g., highly water-soluble therapeutic drug compounds), the modified therapeutic drug compounds of the invention offer advantages associated with relatively slow clearance. The modified therapeutic drug compounds of the invention also provide for distribution properties after administration to a subject that may differ significantly and advantageously compared to the unmodified therapeutic drug compounds.

Representative therapeutic drugs useful in making the modified therapeutic drug compounds of the invention include camptothecin and its derivatives, paclitaxel and its derivatives including docetaxel, doxorubicin, podophyllotoxin and its derivatives including etoposide (anticancer); flucanazole (antifungal); penicillin G, penicillin V (antibacterial); hydralazine, candesartan, and carvediol (anti-hypertensives); isoxicam (anti-inflammatory); metformin (antidiabetic); lazabemide (antiparkinsonian); lamivudine (antiviral); fluoxetine (antidepressant); hydroxyzine (antihistaminic); procainamide hydrochloride (antiarrhythmic); probucol (antihyperlipoproteinemic); azathioprine and cyclosporine (immunosuppressive); danazol (reproductive health); and bosentan (respiratory). It is to be understood that those biologically active materials not specifically mentioned, but having a suitable reactive functional group, for example, including, but not limited to, hydroxyl group, amino group, mercapto or thiol group, or carboxyl group are also intended and are within the scope of the present invention.

In one embodiment, the invention provides cholesterol-modified anti-cancer therapeutic drug compounds in which the cholesterol moiety is covalently coupled to the anti-cancer therapeutic drug moiety through a linker moiety. These cholesterol-modified anti-cancer therapeutic drug compounds have the following formula:

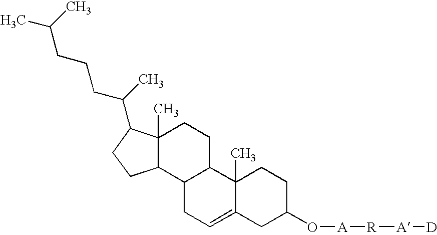

or a stereoisomer, tautomer, or pharmaceutically acceptable salt thereof, wherein A and A′ are independently selected from the group consisting of

(a) —S(═O)—,

(b) —SO₂—,

(c) —C(═O)—

(d) —C(═O)O—,

(e) —C(═O)NR₁—,

(f) —C(═O)OC(═O)—,

(g) —P(═O)(OR₁)O—,

(h) —P(═O)(NR₁)O—,

(i) —SO₂O—,

(j) —S(═O)NR₁—, and

(k) —SO₂NR₁—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl;

R is a divalent radical selected from the group consisting of

(a) substituted or unsubstituted alkylene,

(b) substituted or unsubstituted heteroalkylene,

(c) substituted or unsubstituted cycloalkylene,

(d) substituted or unsubstituted arylene,

(e) amino acid,

(f) peptide,

(g) saccharide, and

(h) alkylene oxide oligomer; and

D is an anti-cancer therapeutic agent moiety.

In one embodiment, the anti-cancer therapeutic agent moiety is selected from a paclitaxel moiety, docetaxel moiety, a camptothecin moiety, and derivatives thereof. Suitable anti-cancer therapeutic agent moieties include a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

In one embodiment, D has the formula

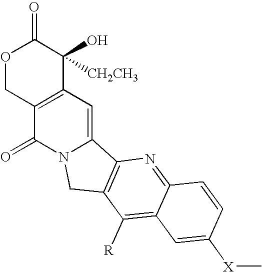

wherein R is selected from the group consisting of H and CH₂CH₃, and X is selected from the group consisting of O and NH.

In one embodiment, D has the formula

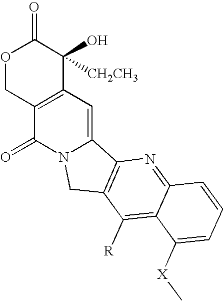

wherein R is selected from the group consisting of H and CH₂CH₃, and X is selected from the group consisting of O and NH.

In one embodiment, D has the formula

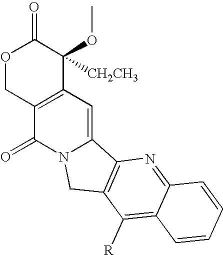

wherein R is selected from the group consisting of H and CH₂CH₃.

In one embodiment, D has the formula

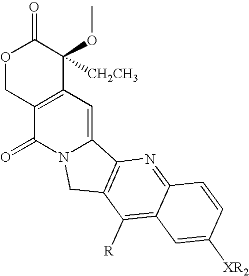

wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

In one embodiment, D has the formula

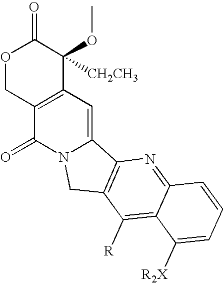

wherein R is selected from the group consisting of H and CH₂CH₃; X is selected from the group consisting of O and NH; R₂ is selected from the group consisting of H, acyl, alkyl, branched alkyl, substituted or unsubstituted cycloalkyl, substituted or unsubstituted aryl, and substituted or unsubstituted aralkyl.

In one embodiment, A is C(═O)—, A′ is —C(═O)—, and R is —(CRₐR_{b})ₘ-, wherein m is 1, 2, or 3, and Rₐ and R_{b} are independently selected from the group consisting of H, CH₃, and taken together with the carbon atom to which they are attached form a 4 to 6-membered substituted or unsubstituted carbon ring.

In another embodiment, the invention provides cholesterol-modified anti-cancer therapeutic drug compounds having the formula:

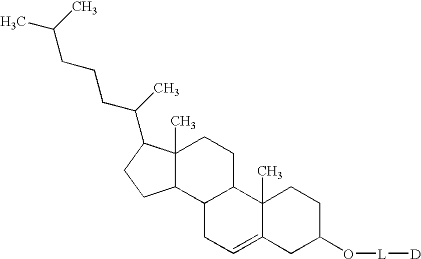

or a stereoisomer, tautomer, or pharmaceutically acceptable salt thereof, wherein L is selected from the group consisting of

(a) —S(═O)—,

(b) —SO₂—,

(c) —C(═O)—

(d) —C(═O)OC(═O)—,

(e) —P(═O)(OR₁)—, and

(f) —P(═O)(NR₁)—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl; and

D is an anti-cancer therapeutic agent moiety.

In one embodiment, the anti-cancer therapeutic agent moiety is selected from a paclitaxel moiety, docetaxel moiety, a camptothecin moiety, and derivatives thereof. Suitable anti-cancer therapeutic agent moieties include a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

In one embodiment, D has the formula

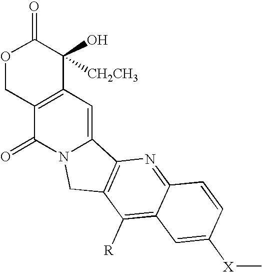

wherein R is selected from the group consisting of H and CH₂CH₃, and X is selected from the group consisting of O and NH.

In one embodiment, D has the formula

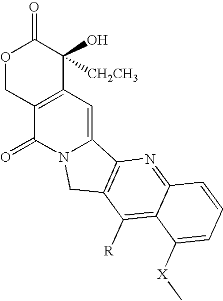

wherein R is selected from the group consisting of H and CH₂CH₃, and X is selected from the group consisting of O and NH.

In one embodiment, D has the formula

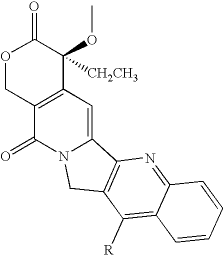

wherein R is selected from the group consisting of H and CH₂CH₃.

In one embodiment, D has the formula

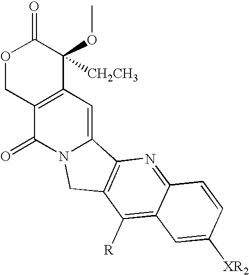

wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

In one embodiment, D has the formula

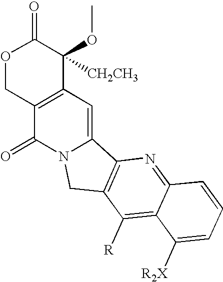

wherein R is selected from the group consisting of H and CH₂CH₃; X is selected from the group consisting of O and NH; R₂ is selected from the group consisting of H, acyl, alkyl, branched alkyl, substituted or unsubstituted cycloalkyl, substituted or unsubstituted aryl, and substituted or unsubstituted aralkyl.

In one embodiment, A is —C(═O)—, A′ is —C(═O)—, and R is —(CRₐR_{b})ₘ-, wherein m is 1, 2, or 3, and Rₐ and R_{b} are independently selected from the group consisting of H, CH₃, and taken together with the carbon atom to which they are attached form a 4 to 6-membered substituted or unsubstituted carbon ring.

Representative cholesterol-modified anti-cancer therapeutic drug compounds of the invention include cholesterol succinate-20-camptothecin, cholesterol succinate-10-(10-hydroxycamptothecin), cholesterol succinate-10-(7-ethyl-10-hydroxycamptothecin), cholesterol formate-20-camptothecin, cholesterol formate-10-(7-ethyl-10-hydroxycamptothecin), cholesterol 3,3-tetramethylene glutaric-20-camptothecin, cholesterol 3,3-tetramethylene glutaric-10-(7-ethyl-10-hydroxycamptothecin, cholesterol 3-methylglutaric-20-camptothecin, cholesterol 3 -methylglutaric-10-(7-ethyl-10-hydroxycamptothecin), 2′-cholesterol succinate paclitaxel, and 2′-cholesterol succinate docetaxel.

In another embodiment, the invention provides bile acid- and bile-acid-derivative-modified anti-cancer therapeutic drug compounds having the formula:

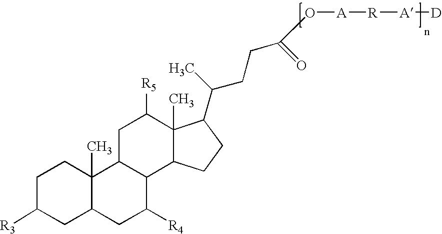

or a stereoisomer, tautomer, or pharmaceutically acceptable salt thereof, wherein

R₃ is OR₆ₐ, and R₄ and R₅ are H; or

R₃ is OR₆ₐ, R₄ is OR_{6b}, and R₅ is H; or

R₃ is OR₆ₐ, R₄ is OR_{6b}, and R₅ is OR_{6c},

wherein R₆ₐ, R_{6b}, and R_{6c} are independently selected from the group consisting of H, substituted or unsubstituted alkyl, substituted or unsubstituted aralkyl, and substituted and unsubstituted acyl;

n is 0 or 1;

A and A′ are independently selected from the group consisting of

(a) —S(═O)—,

(b) —SO₂—,

(c) —C(═O)—

(d) —C(═O)O—,

(e) —C(═O)NR₁—,

(f) —C(═O)OC(═O)—,

(g) —P(═O)(OR₁)O—,

(h) —P(═O)(NR₁)O—,

(i) —SO₂O—,

(h) —S(═O)NR₁—, and

(k) —SO₂NR₁—,

wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl;

R is a divalent radical selected from the group consisting of

(a) substituted or unsubstituted alkylene,

(b) substituted or unsubstituted heteroalkylene,

(c) substituted or unsubstituted cycloalkylene,

(d) substituted or unsubstituted arylene,

(e) amino acid,

(f) peptide,

(g) saccharide, and

(h) alkylene oxide oligomer; and

D is an anti-cancer therapeutic agent moiety.

In one embodiment, the anti-cancer therapeutic agent moiety is selected from a paclitaxel moiety, docetaxel moiety, a camptothecin moiety, and derivatives thereof. Suitable anti-cancer therapeutic agent moieties include a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

In one embodiment, D has the formula

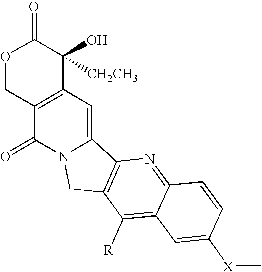

wherein R is selected from the group consisting of H and CH₂CH₃, and X is selected from the group consisting of O and NH.

In one embodiment, D has the formula

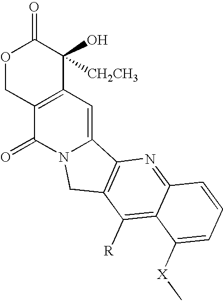

wherein R is selected from the group consisting of H and CH₂CH₃, and X is selected from the group consisting of O and NH.

In one embodiment, D has the formula

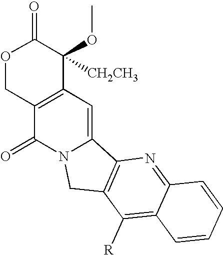

wherein R is selected from the group consisting of H and CH₂CH₃;

In one embodiment, D has the formula

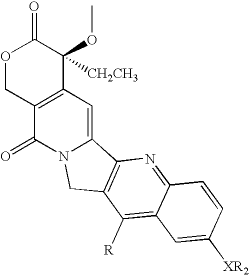

wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

In one embodiment, D has the formula

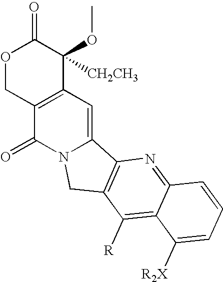

wherein R is selected from the group consisting of H and CH₂CH₃; X is selected from the group consisting of O and NH; R₂ is selected from the group consisting of H, acyl, alkyl, branched alkyl, substituted or unsubstituted cycloalkyl, substituted or unsubstituted aryl, and substituted or unsubstituted aralkyl.

In one embodiment, A is —(═O)—, A′ is —(═O)—, and R is —(CRₐR_{b})ₘ-, wherein m is 1, 2, or 3, and Rₐ and R_{b} are independently selected from the group consisting of H, CH₃, and taken together with the carbon atom to which they are attached form a 4 to 6-membered substituted or unsubstituted carbon ring.

In the compounds above, divalent radical R is selected from the following groups: alkyl (e.g., —(CH₂)ₙ-), substituted alkyl (e.g., —(CHX)ₙ-), branched alkyl (e.g., —CH₂CH(CH₃)CH₂—) (collectively referred to herein as “substituted or unsubstituted alkylene”); cycloalkyl (e.g., 1,4-cyclohexyl or 1,2-cyclopentyl) and substituted cycloalkyl (collectively referred to herein as “substituted or unsubstituted cycloalkylene”); heteroalkyl (e.g., —CH₂OCH₂—) and substituted heteroalkyl (e.g., —CH₂OCH(X)—) (collectively referred to herein as “substituted or unsubstituted heteroalkylene”); aryl (e.g., 1,2-phenyl or 1,4-phenyl) and substituted aryl (collectively referred to herein as “substituted or unsubstituted arylene); aralkylene and substituted aralkylene; amino acid; peptide; polypeptide; protein; mono-, di- or polysaccharide; oligomer of an alkylene oxide, poly(ethylene oxide), poly(propylene oxide), and poly(ethylene oxide)-poly(propylene oxide) oligomers.

Representative bile acid-modified anti-cancer therapeutic drug compounds of the invention include lithocholic-20-camptothecin, lithocholic-10-(7-ethyl-10-hydroxycamptothecin), and lithocholic-10-(10-hydroxycamptothecin).

Representative bile-acid-derivative-modified anti-cancer therapeutic drug compounds of the invention include 3-benzyl lithocholic-20-camptothecin, 3-benzyl lithocholic-10-(7-ethyl-10-hydroxycamptothecin), and 3 -benzyl lithocholic-10-(10-hydroxycamptothecin).

As used herein, the term “alkyl” refers to straight chain and branched alkyl groups, typically having from 1 to 20 carbon atoms. Cycloalkyl groups include monocyclic and polycyclic alkyl groups, monocyclic alkyl groups typically having from about 3 to about 8 carbon atoms in the ring.

The term “aryl” refers to monocyclic and polycyclic aromatic compounds having from 6 to 14 carbon or hetero atoms, and includes carbocyclic aryl groups and heterocyclic aryl groups. Representative aryl groups include phenyl, naphthyl, pyridinyl, pyrimidinyl, thiazolyl, indolyl, imidazolyl, furanyl, and the like. As used herein, the term “aryl” includes heteroaryl groups. The term “aralkyl” refers to an alkyl group that is substituted with an aryl group.

The term “acyl” refers to a —C(═O)R group, where R is a substituted or unsubstituted alkyl, substituted or unsubstituted cycloalkyl, substituted or unsubstituted heteroalkyl, substituted or unsubstituted aryl, substituted or unsubstituted heteroaryl, or substituted or unsubstituted aralkyl group.

The term “substituted” refers to a substituent in which one or more hydrogen atoms is replaced with another group such as, for example, alkyl, cycloalkyl, heteroalkyl, aryl, heteroaryl, halogen, hydroxy, amino, thio, and alkoxy.

In another aspect of the invention, methods for making cholesterol-modified therapeutic drug compounds, bile-acid-modified therapeutic drug compounds, and bile-acid-derivative-modified therapeutic drug compounds are provided. A cholesterol moiety, a bile-acid moiety, or a bile-acid-derivative moiety can be covalently coupled to a therapeutic drug compound to form a cholesterol-modified therapeutic drug compound, a bile-acid-modified therapeutic drug compound, or a bile-acid-derivative-modified therapeutic drug compound, respectively.

In one embodiment, a hydroxyl group of cholesterol, a bile acid, or a bile-acid-derivative may be directly coupled with a carboxyl group of a therapeutic drug compound to form a cholesterol-modified therapeutic drug compound, a bile-acid-modified therapeutic drug compound, or a bile-acid-derivative-modified therapeutic drug compound, respectively.

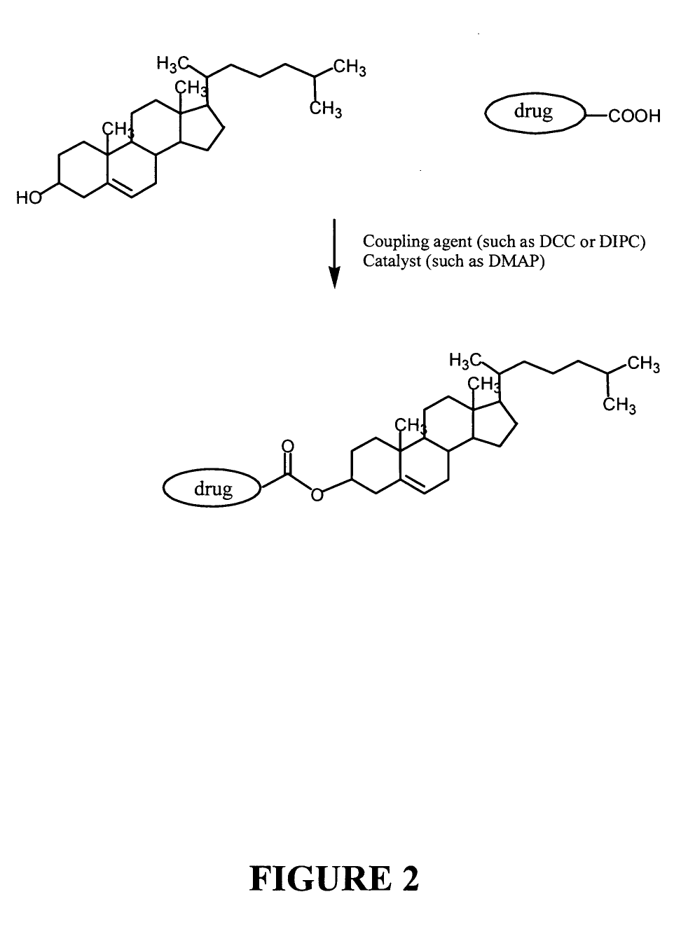

In a representative embodiment, a hydroxyl group of cholesterol is directly coupled with a carboxyl group of a therapeutic drug to form a cholesterol-modified therapeutic drug compound. Such a method is illustrated schematically in FIG. 2.

In another embodiment, cholesterol, a bile acid, or a bile-acid-derivative may be functionalized at the hydroxyl group with a reagent, for example, 2-chloroacetic acid, succinic acid anhydride, phthalic anhydride, isophthalic acid, terephthalic acid, epichlorohydrin, phosphorous oxychloride, alkyl dichlorophosphate, aryl dichlorophosphate, alkyl phosphonic dichloride, aryl phosphonic dichloride, chlorosulfonic acid, or 4-isocyanatobenzoyl chloride. The functional group added to the cholesterol, bile acid, or bile-acid-derivative may be, for example, a carboxyl group (—COOH), oxiranyl group (—CH(O)CH₂), phosphoric chloride group (—P(O)ORCl), phosphonic chloride group (—P(O)RCl), chlorosulfonic group (—SO₂C), isocyanato group (—N═C═O), carbonyl chloride group (—COCl). The resulting carboxyl group, oxiranyl group, isocyanato group, or acid chloride group can then be reacted with a therapeutic drug or functionalized therapeutic drug to provide a cholesterol-modified therapeutic drug compound, a bile-acid-modified therapeutic drug compound, or a bile-acid-derivative-modified therapeutic drug compound, respectively.

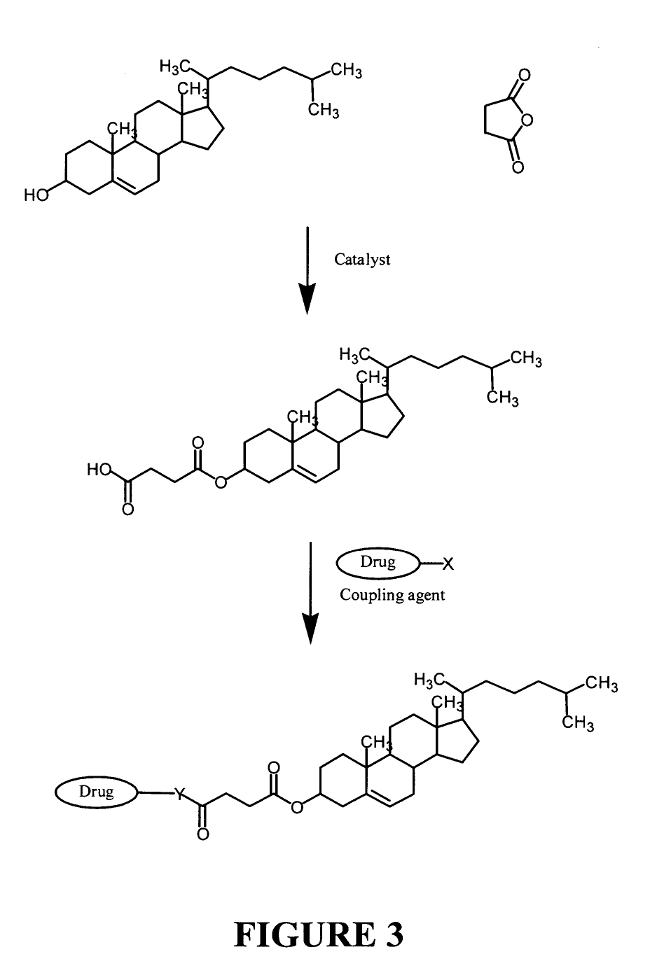

In a representative embodiment, cholesterol is reacted with succinic acid anhydride to form cholesterol succinic acid which couples with the hydroxyl, amine, or carboxyl group of a therapeutic drug to form a cholesterol-modified therapeutic drug compound. Such a method is illustrated schematically in FIG. 3. In FIG. 3, representative X groups include is OH, NH₂, NHR, SH, or CO₂H, and representative Y groups include O, NH, NHR, S, and C(═O)O.

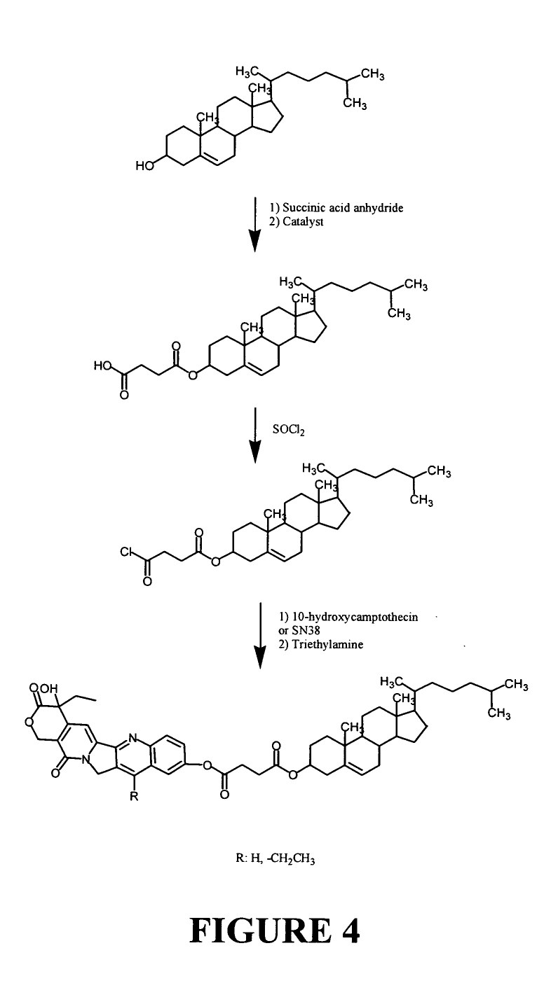

The syntheses of representative cholesterol/bile-acid/bile-acid-derivative-modified therapeutic drug compounds of the invention are illustrated in FIGS. 4-9 and described in Examples 1 to 13.

FIG. 4 illustrates the preparation of cholesterol succinate 10-hydroxycamptothecin and cholesterol succinate 7-ethyl-10-hydroxycamptothecin (SN38) compounds. A free carboxyl group is attached to the hydroxyl group of cholesterol using succinic acid anhydride and a catalyst such as a Lewis acid (e.g., aluminum trichloride). The free carboxyl group is converted to a carbonyl chloride group, which is then coupled to the hydroxyl group at C-10 of 10-hydroxycamptothecin or of 7-ethyl-10-hydroxycamptothecin (SN38) in the presence of a base such as triethylamine to provide cholesterol-modified 10-hydroxycamptothecin and cholesterol-modified SN38. The preparation of cholesterol succinate-10-SN38 is described in Example 1.

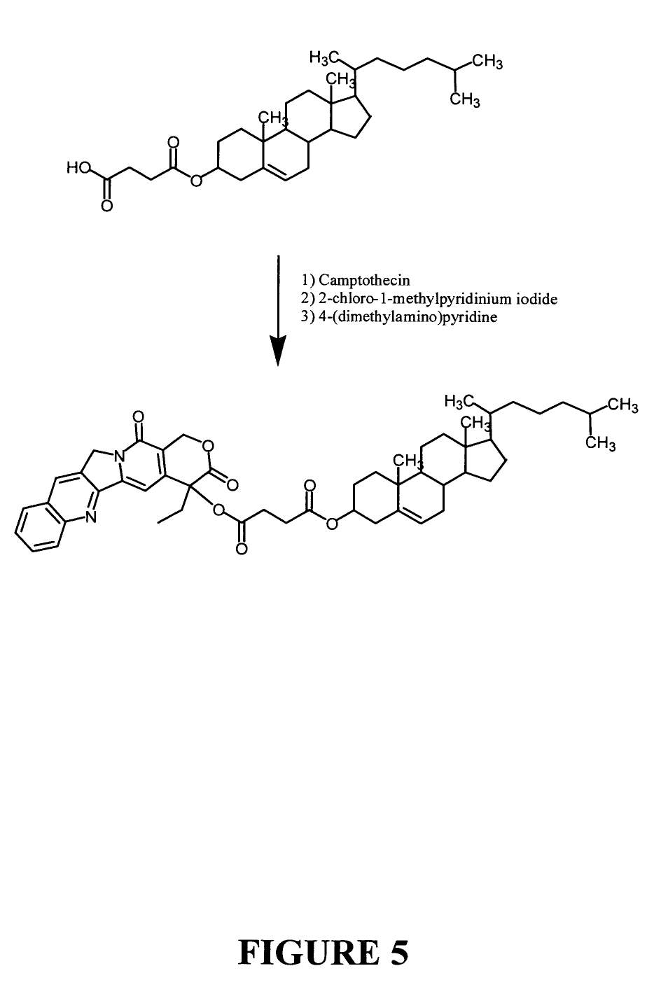

FIG. 5 illustrates the preparation of cholesterol succinate-20-camptothecin. The carboxyl group of cholesterol succinic acid is activated with 2-chloro-1-methylpyridinium iodide in the presence of 4-(methylamino)pyridine, and then coupled with the hydroxyl group of camptothecin to form cholesterol succinate-20-camptothecin. The preparation of cholesterol succinate-20-camptothecin is described in Example 2.

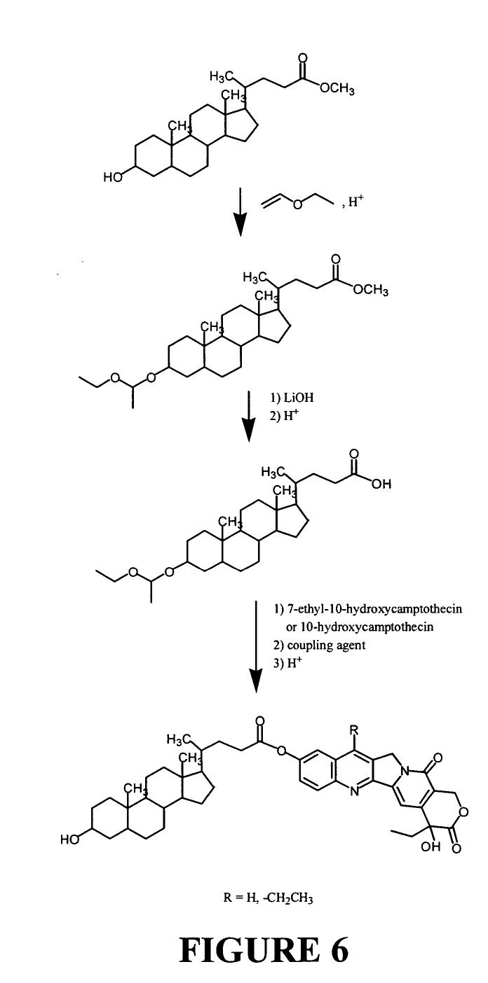

FIG. 6 illustrates the preparation of lithocholic acid-modified 10-hydroxycamptothecin and lithocholic acid-modified SN38. Lithocholic acid methyl ester is used as a starting material. The hydroxyl group at C-3 of lithocholic acid methyl ester is protected by formation of an ethoxyethoxyl group using ethyl vinyl ether. The protected 3-(1-ethoxyethoxyl) lithocholic acid methyl ester is treated with lithium hydroxide (or other suitable alkali hydroxide) to convert the methyl carboxylate group to a free carboxyl group, which is then coupled with the hydroxyl group at C-10 of 10-hydroxycamptothecin or SN38. The protecting group is removed with hydrochloric acid. The preparation of lithocholic-10-(7-ethyl-10-hydroxycamptothecin) is described in Example 9.

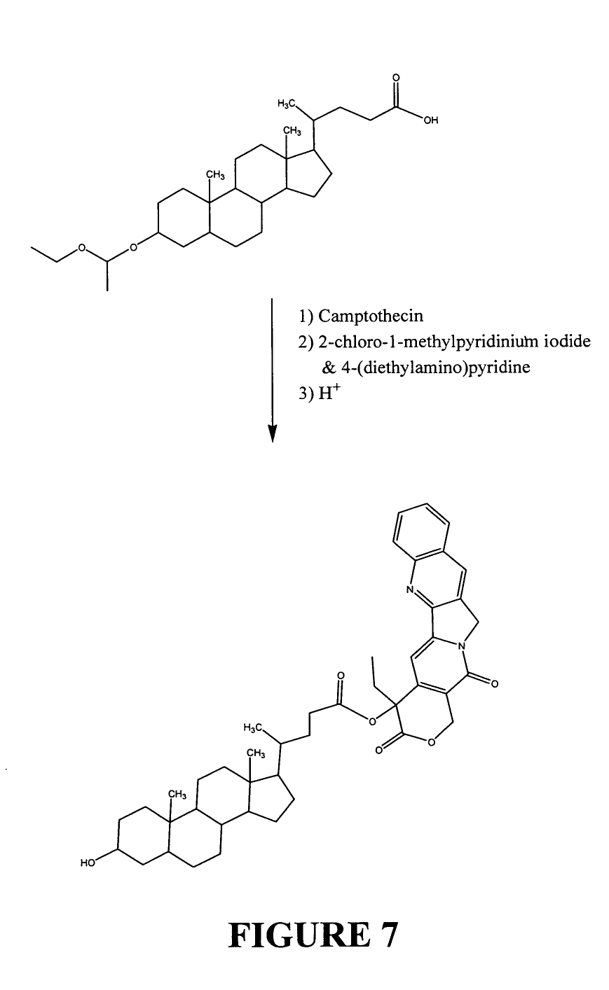

FIG. 7 illustrates the preparation of lithocholic acid-modified camptothecin. The free carboxyl group of 3-(1-ethoxyethoxyl) lithocholic acid is coupled to the hydroxyl group of camptothecin in the presence of a coupling agent, 2-chloro-1-methylpyridinium, and a base, 4-(dimethylamino)pyridine. The protecting group, ethoxyethoxyl group, is removed with hydrochloric acid. The preparation of lithocholic-20-camptothecin is described in Example 10.

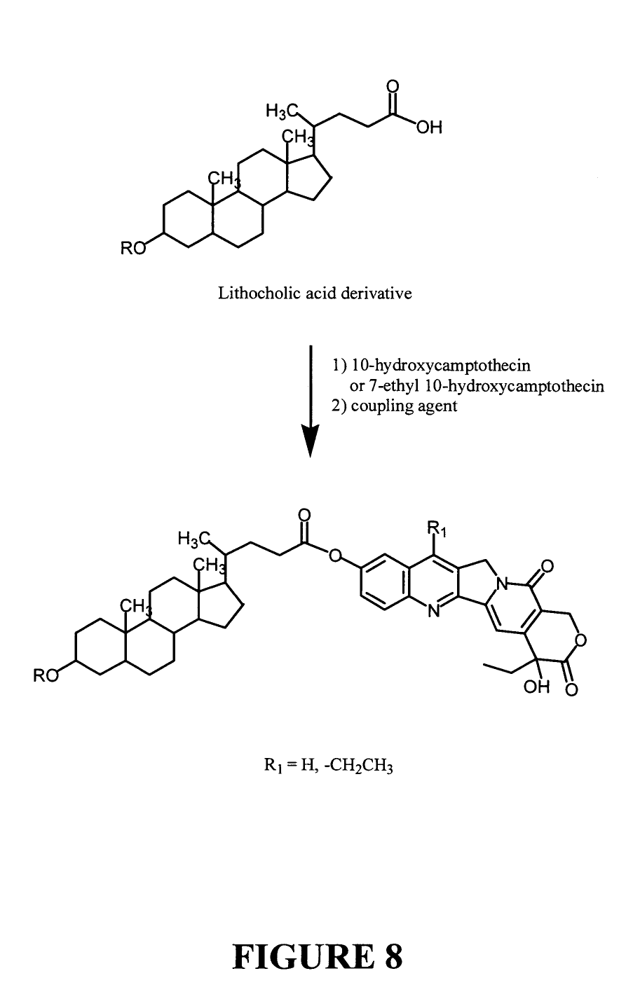

FIG. 8 illustrates the preparation of lithocholic-acid-derivative-modified 10-hydroxycamptothecin and a lithocholic-acid-derivative-modified SN38. The free carboxyl group of a lithocholic-acid derivative is directly conjugated with the hydroxyl group at C-10 of 10-hydroxycamptotheicn or SN38 in the presence of a coupling agent, such as DCC (N,N,-dicyclohexylcarbodiimide), and a catalyst, such as DMAP 4 -(dimethylamino)pyridine). In FIG. 8, R is selected from n-alkyl, branched alkyl, substituted alkyl, cycloalkyl or substituted cycloalkyl, aryl or substituted aryl, aralkyl or substituted aralkyl, allyl or substituted allyl, benzyl or substituted benzyl, acyl, alkyl phosphate, alkyl phosphonate, aryl phosphate, aryl phosphonate, alkyl sulfate, alkyl sulfonate, aryl sulfate, and aryl sulfonate.

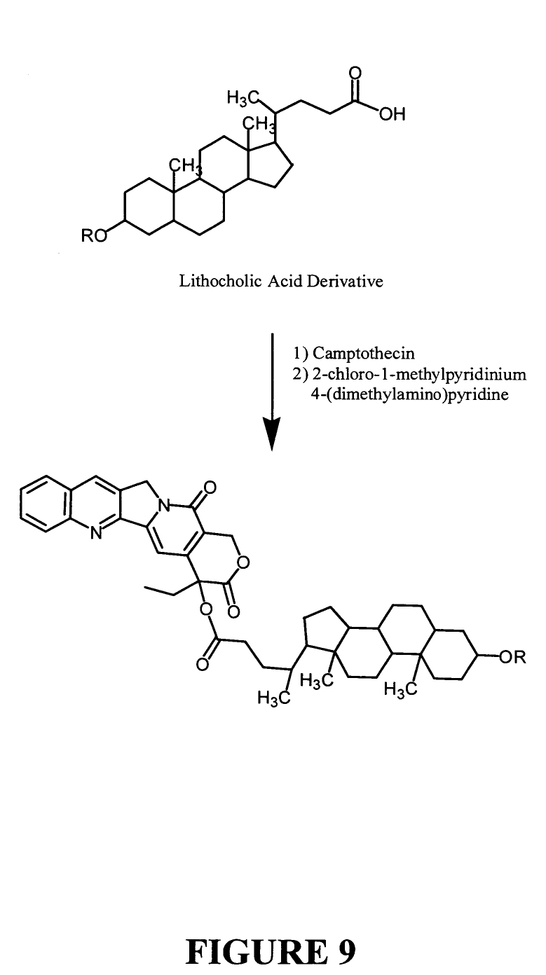

FIG. 9 illustrates the preparation of a lithocholic-acid-derivative-modified camptothecin. The free carboxyl group of a lithocholic-acid derivative couples with the hydroxyl group of camptothecin in the presence of a coupling agent, 2-chloro-1-methylpyridinium, and a base, 4-(dimethylamino)pyridine. In FIG. 9, R is selected from n-alkyl, branched alkyl, substituted alkyl, cycloalkyl or substituted cycloalkyl, aryl or substituted aryl, aralkyl or substituted aralkyl, allyl or substituted allyl, benzyl or substituted benzyl, acyl, alkyl phosphate, alkyl phosphonate, aryl phosphate, aryl phosphonate, alkyl sulfate, alkyl sulfonate, aryl sulfate, and aryl sulfonate. The preparation of a representative lithocholic-acid-derivative-modified camptothecin, 3-benzyl lithocholic-20-camptothecin, is described in Example 11.

In another aspect, the present invention provides compositions that include the compounds of the invention. The compositions include one or more compounds of the invention, optionally one or more additional therapeutic agents, and a lipophilic medium. In one embodiment, a cholesterol/bile-acid/bile-acid-derivative-modified therapeutic drug compound is dissolved in the lipophilic medium. The lipophilic medium (or carrier) of the composition can be any one of a variety of lipophilic mediums including, for example, oils. In one embodiment, the lipophilic medium includes a tocopherol (e.g., α-tocopherol). Representative oils useful as the lipophilic medium include the following:

fatty acids and esters thereof, including carboxylic acids of various chain lengths, mostly straight chain, but which could be branched, examples of which include capric, caprylic, caproic, lauric, myristic, stearic, oleic, linoleic, behenic, and as well as saturated or unsaturated fatty acids and esters;

fatty acids esterified with glycerin to form mono-, di-, or triglycerides, which can be synthetic or derived from natural sources, including, but not limited to, for example, glycerides such as soybean oil, cottonseed oil, rapeseed oil, fish oil, castor oil, Capmul MCM, Captex 300, Miglyol 812, glyceryl monooleate, triacetin, acetylated monoglyceride, tristearin, glyceryl behenate, and diacetyl tartaric acid esters of monoglycerides;

glycerides conjugated to other moieties, such as polyethylene glycol (for example, Labrasol, Labrafac, Cremophor EL);

phospholipids, either natural or synthetic, such as dimyristyl phosphatidylcholine, egg lecithin, and pegylated phospholipids;

other fatty esters including fatty alcohols (myristyl myristate, isopropyl palmitate), or sugars (sorbitan monooleate, SPAN 80, Tween 80, sucrose laurate);

fatty alcohols such as stearyl alcohol, lauryl alcohol, benzyl alcohol, or esters or ethers thereof, such as benzyl benzoate;

fat-soluble vitamins and derivatives, for example, vitamin E (including all of the tocopherols and tocotrienols, and tocopherol and tocotrienol derivatives, such as vitamin E succinate, vitamin E acetate, and vitamin E succinate polyethylene glycol (TPGS)).

Organic co-solvents can also be used in the compositions, optionally in combination with water, including for example, ethanol, polyethylene glycol, propylene glycol, glycerol, N-methyl pyrrolidone, and dimethyl sulfoxide.

The solubility of two representative cholesterol-modified camptothecin compounds of the invention is compared to the solubility of unmodified camptothecin in several mediums in Example 14. The data show that the cholesterol-modified compounds have better solubility in organic solvents than the parent compound, camptothecin.

In a further aspect, the invention provides emulsion, microemulsion, and micelle formulations that include a compound of the invention. Methods for making the emulsions, microemulsions, and micelle formulations are also provided. As used herein, the term “emulsion” refers to a colloidal dispersion of two immiscible liquids, such as an oil and water, in the form of droplets, whose diameter, in general, are between 0.1 and 3.0 microns and that is typically optically opaque, unless the dispersed and continuous phases are refractive index matched. Such systems possess a finite stability, generally defined by the application or relevant reference system, which may be enhanced by the addition of amphiphilic molecules or viscosity enhancers.

The term “microemulsion” refers to a thermodynamically stable isotropically clear dispersion of two immiscible liquids, such as an oil and water, stabilized by an interfacial film of surfactant molecules. A microemulsion has a mean droplet diameter of less than 200 nm, in general between 10-50 nm.

In the absence of water, mixtures of oil(s) and non-ionic surfactant(s) form clear and isotropic solutions that are known as self-emulsifying drug delivery systems (SEDDS) and can be used to improve lipophilic drug dissolution and oral absorption.

The emulsion and microemulsion formulations include an oil phase and an aqueous phase. The emulsion or microemulsion can be an oil-in-water emulsion or a water-in-oil emulsion.

In one embodiment, the compound is present in the formulation in an amount from about 0.005 to about 3.0 weight percent based on the total weight of the formulation. In one embodiment, the compound is present in the formulation in an amount from about 0.01 to about 2.5 weight percent based on the total weight of the formulation. In one embodiment, the compound is present in the formulation in an amount from about 0.1 to about 1.5 weight percent based on the total weight of the formulation.

In one embodiment, the lipophilic medium is present in the formulation in an amount from about 2 to about 20 weight percent based on the total weight of the formulation. In one embodiment, the lipophilic medium is present in the formulation in an amount from about 4 to about 12 weight percent based on the total weight of the formulation. In one embodiment, the lipophilic medium is present in the formulation in an amount from about 6 to about 10 weight percent based on the total weight of the formulation.

In one embodiment of the emulsion or microemulsion, the lipophilic medium includes a tocopherol, and the aqueous medium is water.

In addition to the compounds of the invention, the emulsion or microemulsion formulations can include other components commonly used in emulsions and microemulsions, and, in particular, components that are used in pharmaceutical emulsions and microemulsions. These components include, for example, surfactants and co-solvents. Representative surfactants include nonionic surfactants such as surface active tocopherol derivatives and surface active polymers.

Suitable surface active tocopherol derivatives include tocopherol polyethylene glycol derivatives, such as vitamin E succinate polyethylene glycol (e.g., d-α-tocopherol polyethylene glycol 1000 succinate, TPGS), which is a vitamin E derivative in which a polyethylene glycol is attached by a succinic acid ester at the hydroxyl of vitamin E. As used herein, “vitamin E succinate polyethylene glycol” includes vitamin E succinate polyethylene glycol and derivatives of vitamin E polyethylene glycol having various ester and ether links. TPGS is a non-ionic surfactant (HLB=16-18). TPGS is reported to inhibit P-glycoprotein, a protein that contributes to the development of multi-drug resistance. Embodiments of the formulations of the invention that include TPGS therefore include a P-glycoprotein inhibitor. Surface active tocopherol derivatives (e.g., TPGS) can be present in the formulations of the invention in an amount from about 1 to about 10 weight percent, about 2 to about 6 weight percent, or about 5 weight percent, based on the total weight of the formulation.

Suitable nonionic surfactants include block copolymers of ethylene oxide and propylene oxide known as POLOXAMERS or PLURONICS. These synthetic block copolymers of having the general structure: H(OCH₂CH₂)ₐ(OC₃H₆)_{b}(OCH₂CH₂)ₐOH. The following variants based on the values of a and b are commercially available from BASF Performance Chemicals (Parsippany, N.J.) under the trade name PLURONIC and consist of the group of surfactants designated by the CTFA name of POLOXAMER 108, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403. For the most commonly used POLOXAMERS 124, 188, 237, 338, and 407 the values of a and b are 12/20, 79/28, 64/37, 141/44 and 101/56, respectively. In one embodiment the nonionic surfactant is present in the formulation in an amount from about 0.5 to about 5 weight percent based on the total weight of the formulation.

Co-solvents useful in the formulations include ethanol, polyethylene glycol, propylene glycol, glycerol, N-methylpyrrolidone, dimethylamide, and dimethylsulfoxide, among others. Polyethylene glycol (PEG) is a hydrophilic, polymerized form of ethylene glycol, consisting of repeating units having the chemical structure: (—CH₂CH₂O—). The general formula for polyethylene glycol is H(OCH₂CH₂)ₙOH. The molecular weight ranges from 200 to 10,000. Such various forms are described by their molecular weights, for example, PEG-200, PEG-300, PEG-400, and the like.

Representative emulsions including cholesterol-modified therapeutic drug compounds are described in Example 15.

In vitro cytotoxicities of representative cholesterol-modified therapeutic drug compounds are described in Example 16.

In a further aspect, the invention provides micelle formulations that include a compound of the invention, one or more surfactants, one or more solvents, and an aqueous phase. Micelles are organized aggregates of one or more surfactants in solution. In one embodiment, the compound is present in the formulation in an amount from about 0.005 to about 3.0 weight percent based on the total weight of the formulation. In one embodiment, the compound is present in the formulation in an amount from about 0.01 to about 2.5 weight percent based on the total weight of the formulation. In one embodiment, the compound is present in the formulation in an amount from about 0.1 to about 1.0 weight percent based on the total weight of the formulation. Suitable surfactants include those noted above, and in the amounts noted above. In one embodiment of the micelle formulation, the surfactant is tocopherol polyethylene glycol succinate (TPGS). Representative micelle formulations including cholesterol-modified therapeutic drug compounds are described in Example 15.

The micelle formulation can also include additional components such as solvents and co-solvents, including those noted above. In one embodiment, the micelle formulation includes a polyethylene glycol and a lower alkyl alcohol (e.g., ethanol). In one embodiment, the solvents and co-solvents are present in an amount from about 2 to about 20 weight percent based on the total weight of the formulation. The micelle, emulsion, and microemulsion formulations include an aqueous phase. In one embodiment, the aqueous phase includes deionized water. In another embodiment, the aqueous phase includes saline. In another embodiment, the aqueous phase is saline buffered with an organic acid (e.g., succinate, citrate).

The invention also provides the use of the compounds of the invention in the manufacture of a medicament. For example, for compounds of the invention that include a therapeutic drug moiety derived from a therapeutic drug compound effective in treating cell proliferative disease, the invention provides the use of such compounds in the manufacture of a medicament for the treatment of cell proliferative disease.

In other aspects, methods for administering a compound of the invention to a subject in need thereof, and methods for treating a condition treatable by administration of a therapeutically effective amount of a compound of the invention are also provided. These methods include the administration of the compounds, compositions, emulsion formulations, microemulsion formulations, and micelle formulations described herein.

In one embodiment, the invention provides a method for treating a condition that is treatable by the parent, unmodified therapeutic drug compound (e.g., a cell proliferative disease such as cancer). In the method, a therapeutically effective amount of a compound of the invention is administered to a subject in need thereof.

In one embodiment, the invention provides a method for treating a cell proliferative disease by administering a compound of the invention having a therapeutic drug moiety derived from a therapeutic drug effective in treating cell proliferative disease. Representative cell proliferative diseases treatable by the compounds of the invention include hematologic cancers, such as leukemia, lymphoma, and myeloma; and nonhematologic cancers, such as solid tumor carcinomas (e.g., breast, ovarian, pancreatic, colon, colorectal, non-small cell lung, and bladder), sarcomas, and gliomas.

Therapeutically effective amounts of the compounds will generally range up to the maximally tolerated dosage, but the concentrations are not critical and may vary widely. The precise amounts employed by the attending physician will vary, of course, depending on the compound, route of administration, physical condition of the patient and other factors. The daily dosage may be administered as a single dosage or may be divided into multiple doses for administration.

The amount of the compound actually administered will be a therapeutically effective amount, which term is used herein to denote the amount needed to produce a substantial beneficial effect. Effective doses may be extrapolated from dose-response curves derived from in vitro or animal model test systems. The animal model is also typically used to determine a desirable dosage range and route of administration. Such information can then be used to determine useful doses and routes for administration in humans or other mammals. The determination of an effective dose is well within the capability of those skilled in the art. Thus, the amount actually administered will be dependent upon the individual to which treatment is to be applied, and will preferably be an optimized amount such that the desired effect is achieved without significant side-effects.

Therapeutic efficacy and possible toxicity of the compounds of the invention can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio LD₅₀ to ED₅₀. Modified therapeutic drug compounds that exhibit large therapeutic indices are particularly suitable in the practice of the methods of the invention. The data obtained from cell culture assays and animal studies may be used in formulating a range of dosage for use in humans or other mammals. The dosage of such compounds lies preferably within a range of circulating concentrations that include the ED₅₀ with little or no toxicity. The dosage typically varies within this range depending upon the dosage form employed, sensitivity of the patient, and the route of administration. Thus, optimal amounts will vary with the method of administration, and will generally be in accordance with the amounts of conventional medicaments administered in the same or a similar form.

The compounds of the invention can be administered alone, or in combination with one or more additional therapeutic agents. For example, in the treatment of cancer, the compounds can be administered in combination with therapeutic agents including, but not limited to, androgen inhibitors, such as flutamide and luprolide; antiestrogens, such as tomoxifen; antimetabolites and cytotoxic agents, such as daunorubicin, fluorouracil, floxuridine, interferon alpha, methotrexate, plicamycin, mecaptopurine, thioguanine, adriamycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin, and idamycin; hormones, such as medroxyprogesterone, estramustine, ethinyl estradiol, estradiol, leuprolide, megestrol, octreotide, diethylstilbestrol, chlorotrianisene, etoposide, podophyllotoxin, and goserelin; nitrogen mustard derivatives, such as melphalan, chlorambucil, methlorethamine, and thiotepa, steroids, such as betamethasone; and other antineoplastic agents, such as live Mycobacterium bovis, dicarbazine, asparaginase, leucovorin, mitotane, vincristine, vinblastine, and taxotere. Appropriate amounts in each case will vary with the particular agent, and will be either readily known to those skilled in the art or readily determinable by routine experimentation.

Administration of the compounds of the invention is accomplished by any effective route, for example, parenteral, topical, or oral routes. Methods of administration include inhalational, buccal, intramedullary, intravenous, intranasal, intrarectal, intraocular, intraabdominal, intraarterial, intraarticular, intracapsular, intracervical, intracranial, intraductal, intradural, intralesional, intramuscular, intralumbar, intramural, intraocular, intraoperative, intraparietal, intraperitoneal, intrapleural, intrapulmonary, intraspinal, intrathoracic, intratracheal, intratympanic, intrauterine, intravascular, and intraventricular administration, and other conventional means. The compounds of the invention having anti-tumor activity can be injected directly into a tumor, into the vicinity of a tumor, or into a blood vessel that supplies blood to the tumor.

The emulsion, microemulsion, and micelle formulations of the invention can be nebulized using suitable aerosol propellants that are known in the art for pulmonary delivery of the compounds.

The compounds of the invention may be formulated into a composition that additionally comprises suitable pharmaceutically acceptable carriers, including excipients and other compounds that facilitate administration of the compound to a subject. Further details on techniques for formulation and administration may be found in the latest edition of “Remington's Pharmaceutical Sciences” (Maack Publishing Co., Easton, Pa.).

Compositions for oral administration may be formulated using pharmaceutically acceptable carriers well known in the art, in dosages suitable for oral administration. Such carriers enable the compositions containing the compounds of the invention to be formulated as tablets, pills, dragees, capsules, liquids, gels, syrups, slurries, suspensions, suitable for ingestion by a subject. Compositions for oral use may be formulated, for example, in combination with a solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents may be added, such as the crosslinked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Compounds for oral administration may be formulated, for example, as push-fit capsules made of gelatin, as well as soft, sealed capsules made of gelatin and a coating such as glycerol or sorbitol. Push-fit capsules may contain the compounds mixed with filler or binders such as lactose or starches, lubricants such as talc or magnesium stearate, and, optionally, stabilizers. In soft capsules, the covalent conjugates may be dissolved or suspended in suitable liquids, such as fatty oils, liquid paraffin, or liquid polyethylene glycol with or without stabilizers.

For topical or nasal administration, penetrants appropriate to the particular barrier to be permeated are typically used in the formulation. Examples of these are 2-pyrrolidone, N-methyl-2-pyrrolidone, dimethylacetamide, dimethyl-formamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur. For topical administration, the composition may be in the form of a transdermal ointment or patch for systemic delivery of the compound and may be prepared in a conventional manner (see, e.g., Barry, Dermatological Formulations (Drugs and the Pharmaceutical Sciences—Dekker); Harry's Cosmeticology (Leonard Hill Books).

For rectal administration, the compositions may be administered in the form of suppositories or retention enemas. Such compositions may be prepared by mixing the compounds with a suitable non-irritating excipient that is solid at ordinary temperatures but liquid at the rectal temperature and will therefore melt in the rectum to release the drug. Suitable excipients include, but are not limited to, cocoa butter and polyethylene glycols.

The amounts of each of these various types of additives will be readily apparent to those skilled in the art, optimal amounts being the same as in other, known formulations designed for the same type of administration.

Compositions containing the compounds of the invention may be manufactured in a manner similar to that known in the art (e.g., by means of conventional mixing, dissolving, granulating, dragee-making, levigating, emulsifying, encapsulating, entrapping or lyophilizing processes). The compositions may also be modified to provide appropriate release characteristics, sustained release, or targeted release, by conventional means (e.g., coating). As noted above, in one embodiment, the compounds are formulated as an emulsion.

Compositions containing the compounds may be provided as a salt and can be formed with many acids, including but not limited to hydrochloric, sulfuric, acetic, lactic, tartaric, malic, and succinic. Salts tend to be more soluble in aqueous or other protonic solvents than are the corresponding free base forms.

After compositions formulated to contain a compound and an acceptable carrier have been prepared, they can be placed in an appropriate container and labeled for use. Thus, in another aspect, the invention provides kits.

Cholesterol/bile-acid/bile-acid-derivative-modified therapeutic drug compounds of the invention are suitable for administration as oil-in-water emulsions and micelle formulations. The compounds provide for high drug loading to enable small volumes for administration.

Emulsions containing cholesterol/bile-acid/bile-acid-derivative-modified camptothecin compounds of the invention provide for enhanced stability of the compound's lactone compared to conventional methods of camptothecin administration. Long plasma half-life is achieved for the cholesterol/bile-acid/bile-acid-derivative-modified camptothecin compounds resulting in prolonged exposure of a tumor to the compounds. Cholesterol/bile-acid/bile-acid-derivative-modified compounds achieve high permeation through lipoidal membranes of tumor cells. Greater anti-tumor response without an increase in toxicity may be provided by the cholesterol/bile-acid/bile-acid-derivative-modified camptothecin compounds of the invention as compared to unmodified camptothecin and currently available camptothecin analogs.

The following examples are provided to illustrate, not limit, the invention.

### EXAMPLES

### Example 1

### The Preparation of a Representative Cholesterol-Modified Camptothecin Compound: Cholesterol succinate-10-(7-ethyl-10-hydroxycamptothecin)

A mixture of cholesteryl hemisuccinate (0.973 g, 2 mmol), thionyl chloride (0.238 g, 2 mmol), and toluene (50 ml) was stirred at room temperature overnight. The solvent and excess thionyl chloride were removed under reduced pressure. The residue was collected, and dissolved in 10 ml of chloroform (solution A). SN38 (0.392 g, 1 mmol) was dissolved in 20 ml of anhydrous N,N-dimethylacetamide (solution B). Solution A was added to solution B with stirring, and then triethylamine (0.202 g, 2 mmol) was added to the mixture of solutions A and B. The mixture was stirred overnight at room temperature. The crude product was purified by column chromatography on silica gel (0.310 g, 36.0%).

¹H NMR (300 MHz, CDCl₃): δ 8.246-8.216 (d, J=9 Hz, 1H), 7.835-7.827 (d, J=2.4 Hz, 1H), 7.649 (s, 1H), 7.590-7.552 (dd, J₁=9.3 Hz, J₂=2.4 Hz, 1H), 5.783-5.301 (q, J₁=131.1 Hz, J₂=16.2 Hz, 2H), 5.388-5.373 (d, J=4.5 Hz, 1H), 5.260 (s, 2H), 4.743-4.634 (m, 1H), 3.818 (s, 1H), 3.191-3.114 (q, J=7.5 Hz, 2H), 3.005-2.959 (t, J=6.9 Hz, 2H), 2.809-2.763 (t, J=6.9, 2H), 2.370-2.344 (d, J=7.8 Hz, 2H), 2.074-0.856 (m, 46H), 0.676 (s, 3H).

MS (Positive ESI): m/z 861.5 (M)⁺.

IR (νₘₐₓ cm⁻¹): 3251.47, 2930.54, 1738.00, 1660.21, 1597.99, 1510.57, 1461.42, 1414.21, 1375.84, 1311.14, 1280.91, 1226.75, 1212.06, 1161.03, 1130.26, 1106.31, 1064.04, 1012.67, 978.68, 919.34, 866.37, 831.18, 809.13, 759.03, 723.10, 665.78.

### Example 2

### The Preparation of a Representative Cholesterol-Modified Camptothecin Compound: Cholesterol succinate-20-camptothecin

A mixture of cholesteryl hemisuccinate (0.380 g, 0.78 mmol), camptothecin (0.271 g, 0.78 mmol), 4-(dimethylamino)pyridine (0.190 g, 1.56 mmol) 2-chloro-1-methylpyridinium iodide (0.2 g, 0.78 mmol), and 25 ml of N,N-dimethylacetamide was stirred at room temperature for 24 hours. The reaction was monitored with thin layer chromatography (TLC). After the reaction was completed, 100 ml of ethyl acetate was poured into the reaction mixture. The mixture was washed with saturated aqueous NaCl (3×100 ml). The ethyl acetate portion was collected and dried over anhydrous MgSO₄. The crude product was purified by column chromatography on silica gel (0.330 g, 52.0%).

¹H NMR (300 MHz, CDCl₃): δ 8.390 (s, 1H), 8.254-8.226 (d, J=8.4 Hz, 1H), 7.952-7.926 (d, J=7.8 Hz, 1H), 7.852-7.804 (t, J=7.2 Hz, 1H), 7.693-7.643 (t, J=7.2 Hz, 1H), 7.308 (s, 1H), 5.715-5.370 (q, J, =86.25 Hz, J₂=17.1 Hz, 2H), 5.285 (s, 2H), 5.065-5.049 (d, 1H), 4.590-4.483 (m, 1H), 2.876-2.776 (m, 2H), 2.684-2.554 (m, 2H), 2.348-0.811 (m, 45H), 0.636 (s, 3H).

MS (Positive ESI): m/z 817.2 (M+H)⁺.

IR (νₘₐₓ cm⁻¹): 2944.02, 2868.23, 1727.21, 1671.92, 1627.82, 1564.75, 1497.82, 1456.14, 1405.64, 1383.10, 1364.23, 1352.29, 1323.14, 1293.91, 1249.69, 1231.97, 1164.36, 1148.01, 1131.29, 1082.62, 1061.72, 1045.25, 989.64, 947.22, 928.70, 909.68, 889.17, 827.02, 812.08, 785.60, 760.53, 752.01, 735.88, 722.38, 698.93, 656.19.

### Example 3

### The Preparation of a Representative Cholesterol-Modified Camptothecin Compound: Cholesterol formate-10-(7-ethyl-10-hydroxycamptothecin)

7-Ethyl-10-hydroxycamptothecin (0.392 g, 1 mmol) was suspended in 200 ml of chloroform at room temperature, followed by the addition of cholesteryl chloroformate (0.450 g, 1 mmol) and 4-dimethylaminopyridine (0.244 g, 2 mmol). The resulting mixture was stirred at reflux temperature for two hours. The reaction was monitored by TLC (50% acetone in hexane). After completion, the mixture was washed with 0.1 N HCl (3×100 ml). The organic layer was dried over anhydrous magnesium sulfate, concentrated under reduced pressure to 10 ml, and precipitated with ether. The precipitated solid was filtered and dried (Yield: 0.262 mg, 32.50%).

¹H NMR (300 MHz, CDCl₃): δ 8.240-8.209 (d, J=9.3 Hz, 1H), 7.928-7.920 (d, J=2.4, 1H), 7.676-7.652 (dd, J₁=7.2 Hz, J₂=2.4 Hz, 1H), 7.644 (s, 1H), 5.782-5.280 (q, J₁=134.1 Hz, J₂=16.5 Hz, 2H), 5.460-5.443 (d (broad), J=5.1 Hz, 1H), 5.261 (s, 1H), 4.709-4.601 (m, 1H), 3.827 (s, 1H), 3.199-3.122 (q, J=7.8 Hz, 2H), 2.550-2.468 (m, 2H), 2.107-1.734 (m, 8H), 1.607-0.858 (m, 39H), 0.694 (s, 3H).

MS (Positive ESI): m/z 805.2 (M)⁺.

IR (νₘₐₓ cm⁻¹): 3369.63, 2945.14, 1764.75, 1656.66, 1606.18, 1554.91, 1507.72, 1468.25, 1382.53, 1318.51, 1238.35, 1187.04, 1156.91, 1107.18, 1049.01, 1031.24, 994.52, 974.12, 946.29, 870.07, 835.78, 821.77, 801.34, 779.48, 750.55, 722.15, 666.73.

### Example 4

### The Preparation of a Representative Cholesterol-Modified Camptothecin Compound: Cholesterol Formate-20-camptothecin

Camptothecin (0.348 g, 1 mmol) was suspended in 200 ml of chloroform at room temperature, followed by the addition of cholesteryl chloroformate (0.90 g, 2 mmol) and 4-(dimethylamino)pyridine (0.488 g, 4 mmol). The resulting mixture was stirred at reflux temperature for 24 hours. The reaction was monitored by TLC (50% acetone in hexane). After completion, the mixture was washed with 0.1 N HCl (3×100 ml). The organic layer was dried over anhydrous magnesium sulfate, concentrated under reduced pressure to 10 ml. The crude product was purified by column chromatography on silica gel (Yield: 0.566 g, 74.30%).

¹H NMR (300 MHz, CDCl₃): δ 8.403 (s, 1H), 8.247-8.219 (d, J=8.4 Hz, 1H), 7.960-7.934 (d, J=7.8 Hz, 1H), 7.878-7.819 (dt, J₁=7.8 Hz, J₂=2.4 Hz, 1H), 7.704-7.651 (dt, J₁=7.8 Hz, J₂=0.9 Hz, 1H), 7.358 (s, 1H), 5.729-5.372 (q, J₁=90 Hz, J₂=17.1 Hz, 2H), 5.375 (brs, 1H), 5.297 (s, 2H), 4.439-4.332 (m, 1H), 2.446-2.411 (m, 2H), 2.336-2.125 (m, 2H), 1.980-0.841 (m, 41H), 0.650 (s, 3H).

MS (Positive ESI): m/z 761 (M)⁺.

IR (νₘₐₓ cm⁻¹): 2934.40, 2867.70, 1742.07, 1672.15, 1620.06, 1562.12, 1505.42, 1457.16, 1440.98, 1403.64, 1368.01, 1352.14, 1319.59, 1271.98, 1251.51, 1231.85, 1190.71, 1156.62, 1131.97, 1074.59, 1057.13, 1041.08, 992.32, 972.83, 953.17, 927.23, 889.34, 831.01, 797.74, 784.64, 756.08, 721.74, 656.72.

### Example 5

### The Preparation of a Representative Cholesterol-Modified Camptothecin Compound: Cholesterol 3,3-tetramethylene glutaric-10-(7-ethyl-10-hydroxycamptothecin)

Preparation of cholesterol 3,3-tetramethylene glutaric acid. A mixture of cholesterol (3.86 g, 10 mmol), 3,3-tetramethylene glutaric acid anhydride (1.69 g, 10 mmol), cesium carbonate (3.26 g, 10 mmol), N,N-dimethylformamide (200 ml) in a 500 ml flask was stirred at room temperature under nitrogen overnight. To the mixture was added 200 ml of ethyl acetate. The mixture was washed with dilute hydrochloric acid (0.1N, 3×100 ml), and then dried over anhydrous MgSO₄. The mixture was filtered and filtrate was collected. The crude product was purified by column chromatography on silica gel (2.80 g, 50.5%).

Preparation of cholesterol 3,3-tetramethylene glutaric-10-(7-ehtyl-10-hydroxycamptothecin). A mixture of cholesterol 3,3-tetramethylene glutaric acid (0.554 g, 1 mmol) prepared as above, 7-ethyl-10-hydroxycamptothecin (0.392 g, 1 mmol), 2-chloro-1-methylpyridinium iodide (0.255 g, 1 mmol), 4-(dimethylamino)pyridine (0.244 g, 2 mmol), and N,N-dimethylformamide (50 ml) was stirred at room temperature overnight. To the mixture was added 100 ml of ethyl acetate. The mixture was washed with aqueous NaCl (3×100 ml), and then dried over anhydrous MgSO₄. The crude product was purified by column chromatography on silica gel (0.298 g, 32.07%).

¹H NMR (300 MHz, CDCl₃): δ 8.255-8.224 (d, J=9.3 Hz, 1H), 7.843-7.835 (d, J=2.4 Hz, 1H), 7.647 (s, 1H), 7.605-7.566 (dd, J₁=9.3 Hz, J₂=2.4 Hz, 1H), 5.791-5.288 (q, J₁=134.5 Hz, J₂=16.2 Hz, 2H), 5.371-5.343 (d, J=8.3 Hz, 1H), 5.269 (s, 2H), 4.716-4.607 (m, 1H), 3.712 (s, 1H), 3.202-3.125 (q, J=7.8 Hz, 2H), 2.901 (s, 2H), 2.620 (s, 2H), 2.352-2.326 (d, J=7.8 Hz, 2H), 2.075-0.853 (m, 54H), 0.665 (s, 3H).

MS (Positive ESI): m/z 929.8 (M+H)⁺.

IR (νₘₐₓ cm⁻¹): 3259.94, 2938.23, 1737.85, 1661.65, 1599.10, 1508.68, 1465.54, 1414.16, 1362.61, 1282.54, 1227.20, 1162.80, 1130.91, 1106.35, 1083.51, 1032.28, 1013.39, 924.93, 865.37, 833.91, 759.03, 723.40, 665.90.

### Example 6

### The Preparation of a Representative Cholesterol-Modified Camptothecin Compound: Cholesterol 3,3-tetramethylene glutaric-20-camptothecin

A mixture of cholesterol 3,3-tetramethylene glutaric acid (0.554 g, 1 mmol) prepared as in Example 5, camptothecin (0.348 g, 1 mmol), 2-chloro-1-methylpyridinium iodide (0.255 g, 1 mmol), 4-(dimethylamino)pyridine (0.244 g, 1 mmol), and N,N-dimethylformamide (50 ml) was stirred at room temperature overnight. To the mixture was added 100 ml of ethyl acetate. The mixture was washed with aqueous NaCl (3×100 ml), and then dried over anhydrous MgSO₄. The crude product was purified by column chromatography on silica gel (0.809 g, 91.4%).

IR (νₘₐₓ cm⁻¹): 2935.49, 2868.40, 1739.65, 1661.33, 1598.41, 1508.57, 1465.81, 1413.35, 1374.12, 1255.24, 1227.82, 1162.82, 1130.68, 1065.05, 1040.29, 1010.61, 926.69, 832.10, 809.58, 785.70, 757.26, 722.61, 665.66.

### Example 7

### The Preparation of a Representative Cholesterol-Modified Camptothecin Compound: Cholesterol 3-methylglutaric-10-(7-ethyl-10-hydroxycamptothecin)

Preparation of cholesterol 3-methylglutaric acid. A mixture of cholesterol (3.86 g, 10 mmol), 3-methylglutaric acid anhydride (2.50 g, 20 mmol), cesium carbonate (3.26 g, 10 mmol), dioxane (200 ml) in a 500 ml flask was stirred at room temperature under nitrogen overnight. The solvent was removed under reduced pressure and residue was collected. To the residue was added 200 ml of ethyl acetate. The mixture was washed with dilute hydrochloric acid (0.1 N, 3×100 ml), and then dried over anhydrous MgSO₄. The mixture was filtered and filtrate was collected. The crude product was purified by column chromatography on silica gel (1.803 g, 35.02%).

Preparation of cholesterol 3 -methylglutaric-10-(7-ethyl-10-hydroxycamptothecin). A mixture of cholesterol 3-methylglutaric acid (0.514 g, 1 mmol) prepared as above, 7-ethyl-10-hydroxycamptothecin (0.392 g, 1 mmol), 2-chloro-1-methylpyridinium iodide (0.255 g, 1 mmol), 4-(dimethylamino)pyridine (0.244 g, 1 mmol), and N,N-dimethylformamide (50 ml) was stirred at room temperature overnight. To the mixture was added 100 ml of ethyl acetate. The mixture was washed with aqueous NaCl (3×100 ml), and then dried over anhydrous MgSO₄. The crude product was purified by column chromatography on silica gel (0.153 g, 17.20%).

¹H NMR (300 MHz, CDCl₃): δ 8.258-8.227 (d, J=9.3 Hz, 1H), 7.848-7.840 (d, J=2.4 Hz, 1H), 7.652 (s, 1H), 7.587-7.548 (dd, J₁=9.3 Hz, J₂=2.4 Hz, 1H), 5.790-5.288 (q, J₁=134.2 Hz, J₂=16.2 Hz, 2H), 5.377-5.362 (d, J=4.5 Hz, 1H), 5.269 (s, 2H), 4.675-4.647 (m, 1H), 3.762 (s, 1H), 3.202-3.126 (q, J=7.5 Hz, 2H), 2.789-2.340 (m, 7H), 2.031-0.812 (m, 49H), 0.674 (s, 3H).

IR (νₘₐₓ cm⁻¹): 3253.29, 2935.04, 2868.14, 1737.36, 1660.46, 1597.65, 1508.59, 1466.01, 1412.51, 1374.09, 1277.93, 1257.37, 1227.33, 1211.67, 1162.75, 1129.75, 1106.52, 1080.18, 1064.80, 1039.93, 1010.42, 976.84, 925.97, 865.34, 831.90, 808.95, 785.17, 757.45, 722.69, 692.35, 665.32.

### Example 8

### The Preparation of a Representative Cholesterol-Modified Camptothecin Compound: Cholesterol 3-Methyglutaric-20-Camptothecin

A mixture of cholesterol 3-methylglutaric acid (0.514 g, 1 mmol) prepared as above in Example 7, camptothecin (0.348 g, 1 mmol), 2-chloro-1-methylpyridinium iodide (0.255 g, 1 mmol), 4-(dimethylamino)pyridine (0.244 g, 1 mmol), and N,N-dimethylformamide (50 ml) was stirred at room temperature overnight. To the mixture was added 100 ml of ethyl acetate. The mixture was washed with aqueous NaCl (3×100 ml), and then dried over anhydrous MgSO₄. The crude product was purified by column chromatography on silica gel (0.321 g, 38.53%).

¹H NMR (300 MHz, CDCl₃): δ 8.348 (s, 1H), 8,187-8,160 (d, J=8.1 Hz, 1H), 7.908-7.880 (d, J=8.4 Hz, 1H), 7.810-7.760 (t, J=7.5 Hz, 1H), 7.647-7.599 (t, J=7.2 Hz, 1H), 7.188 (s, 1H), 5.667-5.337 (q, J₁=81.6 Hz, J₂=17.4 Hz, 2H), 5.246 (s, 3H), 4.563-4.523 (m, 1H), 2.602-2.069 (m, 9H), 1.969-0.807 (m, 44H), 0.612 (s, 3H).

IR (νₘₐₓ cm⁻¹): 2935.14, 2868.15, 1739.33, 1661.57, 1598.41, 1566.65, 1509.01, 1465.70, 1406.93, 1374.02, 1254.54, 1227.83, 1162.80, 1130.60, 1081.72, 1065.00, 1041.01, 1000.51, 926.64, 865.52, 831.93, 809.58, 785.87, 756.25, 722.45, 665.45.

### Example 9

### The Preparation of a Representative Bile Acid-Modified Camptothecin Compound: Lithocholic-10-(7-ethyl-10-hydroxycamptothecin)

Preparation of 3-(1-ethoxyethoxyl) lithocholic acid methyl ester. A solution of lithocholic acid methyl ester (1 mmol), pyridinium p-toluene sulfonate (0.1 mmols), ethyl vinyl ether (10 mmols), and CH₂Cl₂ (15 ml) is stirred under nitrogen at room temperature for approximately four hours. The solvent and excess ethyl vinyl ether are removed under reduced pressure. The residue is dissolved in ethyl ether, and washed with water, and dried over anhydrous MgSO₄. The solvent is removed with vacuum, and the crude product is directly used for next step without further purification.

The 3-(1-ethoxyethoxyl)lithocholic acid methyl ester prepared above is dissolved in ethanol/water (ratio 8:1). An equivalent mole of lithium hydroxide (or other suitable alkali hydroxide) is added to the solution and the resulting mixture is stirred for approximately three hours. The mixture is then carefully acidified with 1N HCl and extracted with ethyl ether. The resulting organic layer is separated, dried over anhydrous MgSO₄. The solvent is removed under reduced pressure. The residue, 3-(1-ethoxyethoxyl) lithocholic acid, is used for next step without further purification.

Preparation of Lithocholic-10-(7-ethyl-10-hydroxycamptothecin). A mixture containing 3-(1-ethoxyethoxyl) lithocholic acid (1 mmol) prepared as above, N,N-dicyclohexylcarbodiimide (1 mmol), 4-(dimethylamino)pyridine (0.1 mmol), 7-ethyl-10-hydroxycamptothecin (1 mmol), and dried N,N-dimethylacetamide (30 ml) is stirred at room temperature overnight. The mixture is added into 100 ml of methyl acetate, and washed with saturated aqueous NaCl. The ethyl acetate portion is collected, and ethyl acetate is removed under reduced pressure. The residue is dissolved in ethanol and acidified with 1N HCl. The mixture is concentrated under reduced pressure and dissolved in ethyl acetate (100 ml). The mixture is washed with DI-water. The ethyl acetate portion is dried over anhydrous MgSO₄. After filtration, the solvent is removed under reduced pressure. The crude product is purified by column chromatography on silica gel.

### Example 10

### The Preparation of a Representative Bile Acid-Modified Camptothecin Compound: Lithocholic-20-camptothecin

A mixture containing 3-(1-ethoxyethoxyl) lithocholic acid (1 mmol) prepared as in Example 9, 2-chloro-1-methylpyridinium iodide (1 mmol), 4-(dimethylamino)pyridine (2 mmol), camptothecin (1 mmol), and dried N,N-dimethylacetamide (30 ml) is stirred at room temperature overnight. The mixture is added into ethyl acetate (100 ml), and the resulting mixture is washed with saturated aqueous NaCl. The ethyl acetate portion is dried over anhydrous MgSO₄, and ethyl acetate is removed under reduced pressure. The residue is dissolved in ethanol and acidified with aqueous 1N HCl. The mixture is concentrated under reduced pressure, and added into the ethyl acetate (100 ml). The resulting mixture is washed with DI-water. The ethyl acetate portion is dried over anhydrous MgSO₄. After filtration, the solvent is removed under reduced pressure. The crude product is purified by column chromatography on silica gel.

### Example 11

### The Preparation of a Representative Bile-Acid-Derivative-Modified Camptothecin Compound: 3 -Benzyl lithocholic-20-camptothecin

Preparation of 3-benzyl lithocholic acid. A mixture of lithocholic acid methyl ester (1 equivalent), benzyl chloride (1 equivalent), and cesium carbonate (1 equivalent) in anhydrous N,N-dimethylformamide is stirred at room temperature overnight. The solvent, N,N-dimethylformamide, is removed by reduced pressure. The ethyl acetate is added into the residue with stirring to dissolve the product, 3-benzyl lithocholic acid methyl ester. The resulting mixture is washed with water (3×100 ml). The ethyl acetate portion is dried with anhydrous MgSO₄. The crude product is purified by column chromatography on silica gel to provide 3-benzyl lithocholic acid methyl ester.

The 3-benzyl lithocholic acid methyl ester prepared above is dissolved in ethanol/water (ratio 8:1). An equivalent mole of lithium hydroxide (or other suitable alkali hydroxide) is added to the solution and the resulting mixture is stirred for approximately 4 hours at room temperature. The mixture is then acidified with 1N HCl and extracted with ethyl ether. The resulting organic layer is separated, dried over anhydrous MgSO₄. The solvent is removed under reduced pressure. The residue, 3-benzyl lithocholic acid, is used for next step without further purification.

Preparation of 3-benzyl lithocholic-20-camptothecin. A mixture containing 3-benzyl lithocholic acid (1 equivalent) prepared as above, 2-chloro-1-methylpyridinium iodide (1 equivalent), 4-(dimethylamino)pyridine (2 equivalent), and camptothecin (1 equivalent) in anhydrous N,N-dimethylacetamide is stirred at room temperature overnight. The mixture is added into ethyl acetate, and the resulting mixture is washed with saturated aqueous NaCl. The ethyl acetate portion is dried over anhydrous MgSO₄, and ethyl acetate is removed under reduced pressure. After filtration, the solvent is removed under reduced pressure. The crude product is purified by column chromatography on silica gel.

### Example 12

### The Preparation of a Representative Cholesterol-Modified Paclitaxel Compound: 2′-Cholesterol succinate paclitaxel

A 250 ml flask is charged with 4.86 grams of cholesteryl hemisuccinate, 2.38 grams of thionyl chloride, and 100 ml of toluene. The mixture is stirred at room temperature overnight. The solvent is removed under reduced pressure at 50° C., and the residue is collected. To the residue is added 8.54 grams of paclitaxel and 150 ml of dried tetrahydrofuran with stirring. Then, 1.11 grams of triethylamine in 50 ml of tetrahydrofuran is added dropwise to the reaction mixture. The mixture is stirred at room temperature overnight. The mixture is filtered and the white powder is washed with ethyl acetate (3×100 ml). The filtrate is collected and concentrated by reduced pressure. The crude product is purified by column chromatography on silica gel.

### Example 13

### The Preparation of a Representative Cholesterol-Modified Docetaxel Compound: 2′-Cholesterol succinate docetaxel

A 250 ml flask is charged with 4.86 grams of cholesteryl hemisuccinate, 8.08 grams of docetaxel, 2.06 grams of dried N,N-dicyclohexylcarbodiimide, 500 mg of 4-(dimethylamino)pyridine, and 150 ml of chloroform. The mixture is stirred at room temperature overnight. The mixture is filtered to remove precipitate and the filtrate is collected. The filtrate is concentrated under reduced pressure. The crude product is purified by column chromatography on silica gel.

### Example 14

### Representative Cholesterol-Modified Therapeutic Drug Compound Solubility

In this example, the solubility of representative cholesterol-modified therapeutic drug compounds of the invention, cholesterol formate-20-camptothecin and cholesterol formate-10-(7-ethyl-10-hydroxycamptothecin), was compared to the solubility of camptothecin in a variety of solvents.

Compounds were dissolved in each solvent under constant stirring and temperature. The solubility results showed that the cholesterol-modified camptothecins have better solubility in the organic solvents than camptothecin (Table 1).

The comparative solubility (mg/g) of camptothecin, cholesterol formate-20-camptothecin, and cholesterol formate-10-(7-ethyl-10-hydroxycamptothecin) in various solvents is shown in Table 1.

TABLE 1
Solubility Comparison of Camptothecin
and Cholesterol formate Camptothecins

| | | | Cholesterol | |
|---|---|---|---|---|
| | | Cholesterol | formate- | Tem- |
| | Camptothecin | formate-20-CPT¹ | 10-SN38² | perature |
| Solvent | (mg/g) | (mg/g) | (mg/g) | (° C.) |
| Vitamin E | 1.96 | >32 | >14 | 65 |
| USP/NF | | | | |
| Soybean Oil | 0.00 | >2 | <1 | r.t. |
| USP | | | | |
| Chloroform | 0.71 | >335 | >40 | r.t. |
| Acetonitrile | 0.09 | <1 | <1 | r.t. |

1 Cholesterol formate-20-Camptothecin

2 Cholesterol formate-10-(7-ethyl-10-hydroxycamptothecin)

### Example 15

### Representative Cholesterol-Modified Therapeutic Drug-Containing Emulsion and Micelle Formulations

In this example, representative emulsion and micelle formulations containing a cholesterol-modified therapeutic drug are described.

A. Cholesterol formate-10-(7-ethyl-10-hydroxycamptothecin) emulsion

Cholesterol formate-10-SN38, prepared as described in Example 3, was dissolved in vitamin E (α-tocopherol) and then emulsified by stir and sonication in the presence of d-α-tocopherol polyethylene glycol 1000 succinate (TPGS), polyethylene glycol (PEG 200), and water to produce an emulsion having the following composition (% by weight):

| | |
|---|---|
| Cholesterol formate-10-SN38 | 0.15% |
| Vitamin E | 10% |
| TPGS | 5% |
| PEG(200) | 3% |
| DI water | 81.85% |

B. Cholesterol formate-20-camptothecin emulsion

Cholesterol formate-20-camptothecin, prepared as described in Example 4, is dissolved in vitamin E and then emulsified by stir and sonication in the presence of TPGS, PEG 200, and DI water to produce an emulsion having the following composition (% by weight):

| | |
|---|---|
| Cholesterol formate-20-CPT | 0.2% |
| Vitamin E | 10% |
| TPGS | 5% |
| PEG (200) | 3% |
| Water | 81.8% |

C. Cholesterol 3-methylglutaric-10-(7-ethyl-10-hydroxycamptothecin) micelle formulation

Cholesterol 3 -methyl glutaric-10-(7-ethyl-10-hydroxycamptothecin) was dissolved in a mixture containing TPGS, PEG 200, and ethanol at about 60° C. with stirring for about 1 hour to form a transparent solution. To this solution was added deionized-water (DI-water). The mixture was stirred for a few minutes to form a transparent micelle solution having the following compositions (% by weight):

| | |
|---|---|
| CMG-SN38* | 0.05% |
| TPGS | 5% |
| Ethanol | 5% |
| PEG 200 | 5% |
| DI-water | 84.95% |

The formulation solution was filtered through a 0.2 μm filter and vialed in sterile glass vials.
*CMG-SN38: Cholesterol 3-methyl glutaric-10-(7-ethyl-10-hydroxycamptothecin).

D. Cholesterol 3-methyl glutaric-20-camptothecin micelle formulation

Cholesterol 3-methyl glutaric-20-camptothecin was dissolved in a mixture containing TPGS, PEG(200), and ethanol at about 60° C. with stirring for about 1 hour to form a transparent solution. To this solution was added deionized-water (DI-water). The mixture was stirred for a few minutes to form a transparent micelle solution having the following compositions (% by weight):

| | |
|---|---|
| CMG-CPT* | 0.1% |
| TPGS | 5% |
| Ethanol | 5% |
| PEG(200) | 5% |
| DI-water | 84.9% |

The formulation solution was filtered through a 0.2 μm filter and vialed in sterile glass vials.
*CMG-CPT: Cholesterol 3-methyl glutaric-20-camptothecin.

### Example 16

### In vitro Cytotoxicity of Representative Cholesterol-Modified Therapeutic Drug Compounds

In this example, the in vitro cytotoxicty of representative cholesterol-modified therapeutic drug compounds of the invention, cholesterol succinate-10-(7-ethyl-10-hydroxycamptothecin), cholesterol 3-methyl glutaric-10-(7-ethyl-10-hydroxycamptothecin), cholesterol formate-10-(7-ethyl-10-hydroxycamptothecin), cholesterol 3,3-methylene-10-(7-ethyl-10-hydroxycamptothecin), cholesterol succinate-20-camptothecin, cholesterol 3-methyl glutaric-20-camptothecin, cholesterol formate-20-camptothecin, and cholesterol 3,3-methylene-20-camptothecin, was compared to the in vitro cytotoxicity of 7-ethyl-10-hydroxycamptothecin (SN38), camptothecin (CPT), irinotecan, and topotecan.

The in vitro cytotoxicity, as measured by GI₅₀ (50% of growth inhibition) values, of cholesterol succinate-10-SN38, cholesterol 3-methyl glutaric-10-SN38, cholesterol formate-10-SN38, cholesterol 3,3-methylene-10-SN38, cholesterol succinate-20-CPT, cholesterol 3-methyl glutaric-20-CPT, cholesterol formate-20-CPT, and cholesterol 3,3-methylene-20-CPT was investigated and compared to the National Cancer Institute (NCI) GI₅₀ values for SN-38, camptothecin, irinotecan, and topotecan in the following cancer cell lines: NCI-H460 (ATCC #HTB-177) (non-small cell lung), HCT-15 (ATCC #CCL-225) (colorectal), HT-116 (ATCC #CCL-247) (colorectal), and SKOV-3 (ATCC #HTB-77) (ovarian).

The study was performed using a solution of the cholesterol-modified compounds in DMSO (1 mM) diluted in the corresponding cell media. The cells were in contact with varying concentrations of the test article for a period of 48 hours. At the end of 48 hours, staining with ALAMAR BLUE was performed to determine the number of viable cells and calculate the degree of cellular growth inhibition as compared to a control group. The percent of inhibition versus concentration was fit to the Hill equation to determine concentration that produces 50% of growth inhibition (GI₅₀).

The sensitivity of the tested cell lines to cholesterol succinate-10-SN38, cholesterol 3-methyl glutaric-10-SN38, cholesterol formate-10-SN38, cholesterol 3,3-methylene-10-SN38, cholesterol succinate-20-CPT, cholesterol 3-methyl glutaric-20-CPT, cholesterol formate-20-CPT, cholesterol 3,3-tetramethylene glutaric-20-CPT, SN38, irinotecan, and topotecan is illustrated in Table 2.

TABLE 2
Comparative drug concentration that
produce 50% cell growth inhibition (GI₅₀).

| | Cell line | | | | | | | |
|---|---|---|---|---|---|---|---|---|
| | H460 | | HCT-15 | | HCT-116 | | SKOV-3 | |
| Compound | (NSCLC) | | (COLON) | | (COLON) | | (OVARIAN) | |
| Cholesterol succinate- | 1.66 | μM | 635 | nM | 339 | nM | 708 | nM |
| 10-SN38 | | | | | | | | |
| Cholesterol 3-methyl | 245 | nM | 2.88 | μM | 263 | nM | poor | |
| glutaric-10-SN38 | | | | | | | | |
| Cholesterol formate- | 10.5 | nM | 1.91 | μM | 631 | nM | 51.38 | nM |
| 10-SN38 | | | | | | | | |
| Cholesterol 3,3- | 871 | nM | 12.6 | μM | 2.00 | μM | 295 | nM |
| tetramethylene | | | | | | | | |
| glutaric-10-SN38 | | | | | | | | |
| Cholesterol succinate- | >5 | μM | >5 | μM | >5 | μM | >5 | μM |
| 20-CPT | | | | | | | | |
| Cholesterol 3-methyl | poor | | poor | | poor | | poor | |
| glutaric-20-CPT | | | | | | | | |
| Cholesterol formate- | poor | | poor | | poor | | poor | |
| 20-CPT | | | | | | | | |
| Cholesterol 3,3- | ˜10 | μM | ˜10 | μM | ˜10 | μM | poor | |
| tetramethylene | | | | | | | | |
| glutaric-20-CPT | | | | | | | | |
| SN38 | 1.4 | nM | 7.9 | nM | 21 | nM | 1.0 | nM |
| (NCI) | | | | | | | | |
| Camptothecin | 16 | nM | 160 | nM | 40 | nM | 25 | nM |
| (NCI) | | | | | | | | |
| Irinotecan | 5.01 | μM | 31.6 | μM | 7.9 | μM | 17 | nM |
| (NCI) | | | | | | | | |
| Topotecan | 19.9 | nM | 501 | nM | 39.8 | nM | 63 | nM |
| (NCI) | | | | | | | | |

SN38: 7-ethyl-10-hydroxycamptothecin;
CPT: camptothecin.

The results in Table 2 illustrates that some of the cholesterol-modified therapeutic drug compounds of the invention provide effective anti-tumor activity.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A compound having the formula 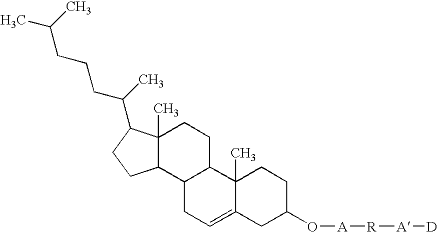
or a stereoisomer, tautomer, or pharmaceutically acceptable salt thereof, wherein
A and A′ are independently selected from the group consisting of
(a) —S(═O)—,
(b) —SO₂—,
(c) —C(═O)—
(d) —C(═O)O—,
(e) —C(═O)NR₁—,
(f) —C(═O)OC(═O)—,
(g) —P(═O)(OR₁)O—,
(h) —P(═O)(NR₁)O—,
(i) —SO₂O—,
(j) —S(═O)NR₁—, and
(k) —SO₂NR₁—,
wherein R₁ is selected from Na⁺, K⁺, H, C₁₋₆ n-alkyl, C₃-₁₂ branched alkyl, substituted or unsubstituted C₃₋₆ cycloalkyl, substituted or unsubstituted aryl, and substituted or unsubstituted aralkyl;
R is a divalent radical selected from the group consisting of
(a) substituted or unsubstituted alkylene,
(b) substituted or unsubstituted heteroalkylene,
(c) substituted or unsubstituted cycloalkylene,
(d) substituted or unsubstituted arylene,
(e) amino acid,
(f) peptide,
(g) saccharide, and
(h) alkylene oxide oligomer; and
D is an anti-cancer therapeutic agent moiety.

2. The compound of claim 1, wherein the anti-cancer therapeutic agent moiety is selected from the group consisting of a paclitaxel moiety, docetaxel moiety, a camptothecin moiety, and derivatives thereof.

3. The compound of claim 1, wherein the anti-cancer therapeutic agent moiety is selected from the group consisting of a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

4. The compound of claim 1, wherein D has the formula 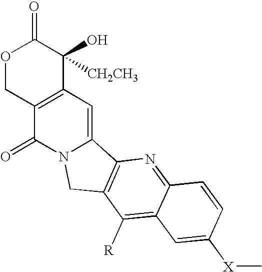
wherein R is selected from the group consisting of H and CH₂CH₃, and X is selected from the group consisting of O and NH.

5. The compound of the claim 1, where D has the formula 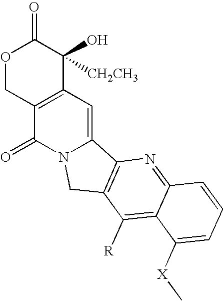
wherein R is selected from the group consisting of H and CH₂CH₃, and X is selected from the group consisting of O and NH.

6. The compound of claim 1, wherein D has the formula 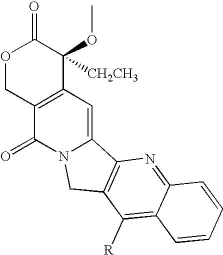
wherein R is selected from the group consisting of H and CH₂CH₃.

7. The compound of claim 1, wherein D has the formula 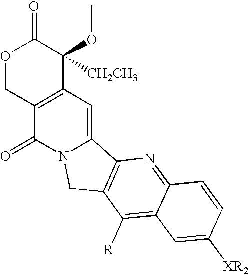
wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

8. The compound of claim 1, wherein D has the formula 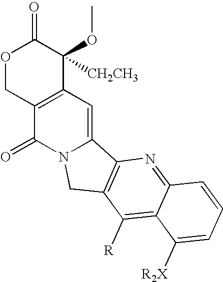
wherein R is selected from the group consisting of H and CH₂CH₃; X is selected from the group consisting of O and NH; R₂ is selected from the group consisting of H, acyl, alkyl, branched alkyl, substituted or unsubstituted cycloalkyl, substituted or unsubstituted aryl, and substituted or unsubstituted aralkyl.

9. The compound of claim 1, wherein A is —C(═O)—, A′ is —C(═O)−, and R is —(CRₐR_{b})ₘ-, wherein m is 1, 2, or 3, and Rₐ and R_{b} are independently selected from the group consisting of H, CH₃, and taken together with the carbon atom to which they are attached form a 4 to 6-membered substituted or unsubstituted carbon ring.

10. A compound having the formula 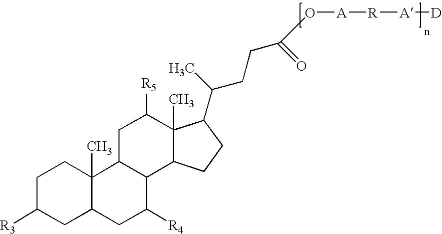
or a stereoisomer, tautomer, or pharmaceutically acceptable salt thereof, wherein
R₃ is OR₆ₐ, and R₄ and R₅ are H; or
R₃ is OR₆ₐ, R₄ is OR_{6b}, and R₅ is H; or
R₃ is OR₆ₐ, R₄ is OR_{6b}, and R₅ is OR_{6c},
wherein R₆ₐ, R_{6b}, and R_{6c}, are independently selected from the group consisting of H, substituted or unsubstituted alkyl, substituted or unsubstituted aralkyl, and substituted and unsubstituted acyl;
n is 0 or 1;
A and A′ are independently selected from the group consisting of
(a) —S(═O)—,
(b) —SO₂—,
(c) —C(═O)—
(d) —C(═O)O—,
(e) —C(═O)NR₁—,
(f) —C(═O)OC(═O)—,
(g) —P(═O)(OR₁)O—,
(h) —P(═O)(NR₁)O—,
(i) —SO₂O—,
(j) —S(═O)NR₁—, and
(k) —SO₂NR₁—,
wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl;
R is a divalent radical selected from the group consisting of
(a) substituted or unsubstituted alkylene,
(b) substituted or unsubstituted heteroalkylene,
(c) substituted or unsubstituted cycloalkylene,
(d) substituted or unsubstituted arylene,
(e) amino acid,
(f) peptide,
(g) saccharide, and
(h) alkylene oxide oligomer; and
D is an anti-cancer therapeutic agent moiety.

11. The compound of claim 10, wherein the anti-cancer therapeutic agent moiety is selected from the group consisting of a paclitaxel moiety, a docetaxel moiety, a camptothecin moiety, and derivatives thereof.

12. The compound of claim 10, wherein the anti-cancer therapeutic agent moiety is selected from the group consisting of a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

13. The compound of claim 10, wherein D has the formula 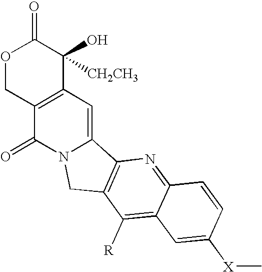
wherein R is selected from the group consisting of H and CH₂CH₃, and X is selected from the group consisting of O and NH.

14. The compound of claim 10, wherein D has the formula 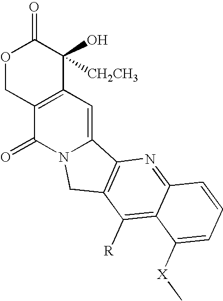
wherein R is selected from the group consisting of H and CH₂CH₃, and X is selected from the group consisting of O and NH.

15. The compound of claim 10, wherein D has the formula 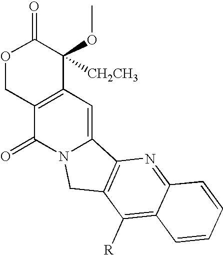
wherein R is selected from the group consisting of H and CH₂CH₃.

16. The compound of claim 10, wherein D has the formula 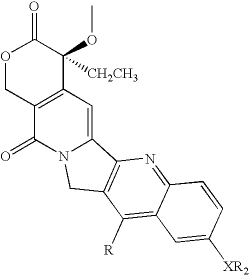
wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

17. The compound of claim 10, wherein D has the formula 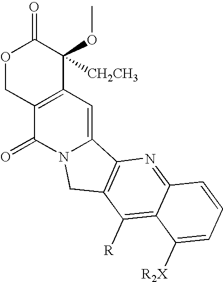
wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

18. The compound of claim 10, wherein A is —C(═O)—, A′ is —C(═O)—, and R is —(CRₐR_{b})ₘ-, wherein m is 1, 2, or 3, and Rₐ and R_{b} are independently selected from the group consisting of H, CH₃, and taken together with the carbon atom to which they are attached form a 4 to 6-membered substituted or unsubstituted carbon ring.

19. A compound having the formula 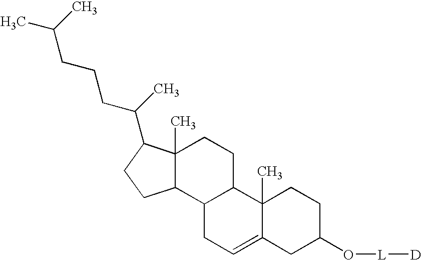
or a stereoisomer, tautomer, or pharmaceutically acceptable salt thereof, wherein L is selected from the group consisting of
(a) —S(═O)—,
(b) —SO₂—,
(c) —C(═O)—
(d) —C(═O)OC(═O)—,
(e) —P(═O)(OR₁)—, and
(f) —P(═O)(NR₁)—,
wherein R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl; and
D is an anti-cancer therapeutic agent moiety.

20. The compound of claim 19, wherein the anti-cancer therapeutic agent moiety is selected from the group consisting of a paclitaxel moiety, a docetaxel moiety, a camptothecin moiety, and derivatives thereof.

21. The compound of claim 19, wherein the anti-cancer therapeutic agent moiety is selected from the group consisting of a camptothecin moiety, a 10-hydroxycamptothecin moiety, a 7-ethyl-10-hydroxycamptothecin moiety, a 9-aminocamptothecin moiety, a 9-amino-7-ethylcamptothecin moiety, a 10-aminocamptothecin moiety, and a 10-amino-7-ethylcamptothecin moiety.

22. The compound of claim 19, wherein D has the formula 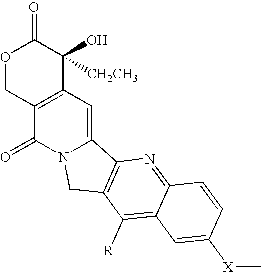
wherein R is selected from the group consisting of H and CH₂CH₃, and X is selected from the group consisting of O and NH.

23. The compound of claim 19, wherein D has the formula 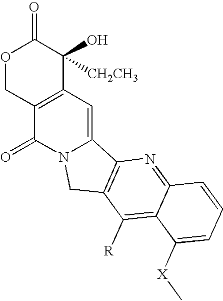
wherein R is selected from the group consisting of H and CH₂CH₃, and X is selected from the group consisting of O and NH.

24. The compound of claim 19, wherein D has the formula 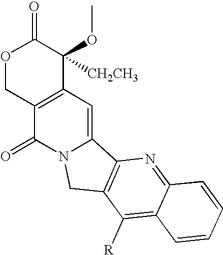
wherein R is selected from the group consisting of H and CH₂CH₃.

25. The compound of claim 19, wherein D has the formula 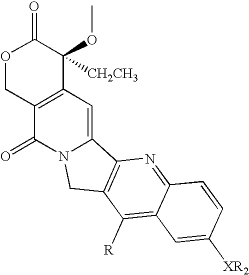
wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

26. The compound of claim 19, wherein D has the formula 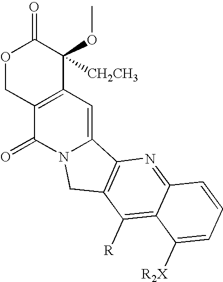
wherein R is selected from the group consisting of H and CH₂CH₃; X is selected from the group consisting of O and NH; and R₂ is selected from the group consisting of H, acyl, alkyl, branched alkyl, substituted or unsubstituted cycloalkyl, substituted or unsubstituted aryl, and substituted or unsubstituted aralkyl.

27. An emulsion, comprising:
(a) a compound of claim 1;
(b) a lipophilic medium; and
(c) an aqueous phase.

28. The emulsion of claim 27, wherein the therapeutic drug moiety is selected from the group consisting of a paclitaxel moiety, a docetaxel moiety, a camptothecin moiety, and derivatives thereof.

29. A micelle formulation, comprising:
(a) a compound of claim 1;
(b) one or more surfactants;
(c) one of more solvents; and
(d) an aqueous phase.

30. The formulation of claim 29, wherein the therapeutic drug moiety is selected from the group consisting of a paclitaxel moiety, a docetaxel moiety, a camptothecin moiety, and derivatives thereof.

31. A method for treating a cell proliferative disease, comprising administering to a subject in need thereof a therapeutically effective amount of a compound of claim 1.

32. The method of claim 31, wherein administering the compound comprises administering an emulsion comprising the compound.

33. The method of claim 31, wherein administering the compound comprises administering a micelle formulation comprising the compound.

34. The method of claim 31, wherein the therapeutic drug moiety is selected from the group consisting of a paclitaxel moiety, a docetaxel moiety, a camptothecin moiety, and derivatives thereof.
